(19) United States
(12) Patent Application Publication
(10) Pub. No.: US 2024/0241130 A1
(43) Pub. Date: Jul. 18, 2024

(54) BIOMARKER FOR PREDICTING RESPONSE TO IMMUNE CHECKPOINT INHIBITOR

(71) Applicant: The University of Tokyo, Bunkyo-ku, Tokyo (JP)

(72) Inventors: Takahiro AMEMIYA, Bunkyo-ku, Tokyo (JP); Masashi HONMA, Bunkyo-ku, Tokyo (JP); Yoshiaki KARIYA, Bunkyo-ku, Tokyo (JP); Hiroshi SUZUKI, Bunkyo-ku, Tokyo (JP)

(21) Appl. No.: 18/684,272

(22) PCT Filed: Aug. 18, 2022
(86) PCT No.: PCT/JP2022/031229

§ 371 Date: Feb. 16, 2024

Related U.S. Application Data
(60) Provisional application No. 63/234,305, filed on Aug. 18, 2021.

(51) Int. Cl. G01N 33/68

(52) U.S. Cl. CPC G01N 33/68; G01N 2800/52

(57) ABSTRACT

An object of the present invention is to provide a method for predicting responsiveness to an immune checkpoint inhibitor and a method for predicting prognosis of a cancer patient who has received treatment with an immune checkpoint inhibitor. According to the present invention, there is provided a method for predicting responsiveness to an immune checkpoint inhibitor by using an amount of an IL-1 signaling pathway molecule in a subject in need of treatment of cancer as an indicator. According to the present invention, there is also provided a method for predicting prognosis of a cancer patient who has received treatment with an immune checkpoint inhibitor, the method comprising predicting prognosis by using an amount of an IL-1 signaling pathway molecule of the subject as an indicator.

### CROSS-REFERENCE TO RELATED APPLICATION

The present application enjoys the benefit of priority from the prior US Patent Application No. 63/234,305 (filing date: Aug. 18, 2021), the entire disclosures of which are incorporated herein by reference.

### TECHNICAL FIELD

The present invention relates to a method for predicting responsiveness to an immune checkpoint inhibitor. The present invention also relates to a method for predicting prognosis of cancer in treatment with an immune checkpoint inhibitor.

### BACKGROUND ART

Cancer is the leading cause of death in Japan, and improvement of the treatment outcome of cancer is an important clinical and social problem. In recent years, while many novel anticancer drugs with various action mechanisms have been developed, particularly with respect to advanced malignant melanoma for which there has been almost no effective treatment method due to the development of immune checkpoint inhibitors, significant improvement in the overall survival rate has been shown and has attracted attention (Non Patent Document 1). At present, in addition to the expansion of application to other cancer types, clinical trials for evaluating the efficacy of various novel regimens such as combination with chemotherapy and molecular target drugs are actively conducted, and it is considered that immune checkpoint inhibitors will become a major therapy for cancer treatment in the future. However, at the same time, it has also been revealed that even such a latest anticancer drug therapy is divided into a patient group with good therapeutic responsiveness and a patient group with poor therapeutic responsiveness, and in order to further improve the therapeutic result of anticancer drug therapy, it is essential to establish personalized medical care for predicting therapeutic responsiveness for each patient and selecting an optimal anticancer drug therapy.

Since immune checkpoint inhibitors, which are rapidly expanding in clinical use, do not directly target tumors but contribute to the immune response of the patient to target tumor shrinkage, the state of both the immune system of the patient and the tumor tissue to be attacked is considered as a factor determining responsiveness to immune checkpoint inhibitors. However, in the evaluation of responsiveness to immune checkpoint inhibitors, stratification based only on the evaluation of tumor tissues is the mainstream, and there is a current situation in which evaluation regarding the immune system of the patient is not sufficiently performed.

In clinical practice, it is recommended to verify the expression of PD-L1 (Programmed cell Death 1-Ligand 1) in a tumor in addition to the presence or absence of a driver gene mutation when determining a treatment policy in the first line of stage 4 non-small cell lung cancer. When the driver mutation is negative, the standard treatment is determined according to the degree of PD-L1 expression in the tumor. Specifically, in the case of PD-L1≥1%, many regimens including a combination therapy of a PD-1/PD-L1 inhibitor and a cytotoxic anticancer drug, a combination therapy of a PD-1 inhibitor and an anti-CTLA-4 (cytotoxic T lymphocyte antigen 4) antibody, and the like can be used as a combined immunotherapy aiming at a synergistic effect by combining a pembrolizumab single agent of a PD-1 (Programmed cell death 1) inhibitor, an atezolizumab single agent of a PD-L1 inhibitor, and therapies with different action mechanisms (Guidelines for Diagnosis and Treatment of Lung Cancer 2020 edited by The Japan Lung Cancer Society). In the case of PD-L1<1%, a combination therapy of a PD-1/PD-L1 inhibitor and a cytotoxic anticancer drug, a combination therapy of a PD-1 inhibitor and an anti-CTLA-4 antibody, and the like can be used. However, when pembrolizumab is added to a chemotherapeutic agent for a non-small cell lung cancer patient having a PD-L1 status of 50% or more, both the overall survival time and the progression-free survival time are prolonged, but the response rate is 47.6% (Non Patent Document 2), and it has been reported that only high expression of PD-L1 in a tumor is not a determinant of treatment.

In the first line treatment for advanced renal cancer including a component of clear cell renal cell carcinoma, treatment selection based on PD-L1 expression in the tumor has not been performed, the prognostic risk according to the standards of the International Metastatic Renal Cell Carcinoma Database Consortium (IMDC) is evaluated, and a combination therapy of ipilimumab and nivolumab is recommended for the intermediate/poor risk group (Guidelines for Diagnosis and Treatment of Renal Cancer 2017 edited by The Japanese Urological Association). In fact, in patients with high expression of PD-L1, the overall survival time is significantly prolonged in the combination group of ipilimumab and nivolumab compared to the sunitinib group, but the combination therapy of ipilimumab and nivolumab is effective regardless of the expression of PD-L1 (Non Patent Document 3), and it cannot be said that the expression of PD-L1 is a clear therapeutic effect predictor at present. As described above, there is a report that there is no significant correlation between the PD-L1 status and the therapeutic effect depending on the cancer type (Non Patent Document 4). Under the current circumstances where the problem of heterogeneity of the PD-L1 molecule in the tumor and the technical problem of immunostaining of PD-L1 itself remain, it is not clear whether expression of PD-L1 on the tumor before treatment is a factor for predicting the therapeutic effect.

### REFERENCE LIST

### Non Patent Documents

Non Patent Document 1: Hodi F S et al, N Engl J Med. 2010; 363(8):711-723.
Non Patent Document 2: Gandhi L et al, N Engl J Med. 2018; 378(22):2078-2092.
Non Patent Document 3: Motzer R I et al, N Engl J Med. 2018; 378(14):1277-1290.
Non Patent Document 4: Ansell S M et al, N Engl J Med. 2015; 372(4):311-9.

### SUMMARY OF THE INVENTION

The present inventors have now found that by analyzing a blood specimen obtained from a tumor-bearing mouse model with a mass spectrometer and analyzing a serum specimen obtained from a cancer patient by an immunological method, responsiveness to an immune checkpoint inhibitor can be predicted at a stage before starting treatment by using the level of an IL-1 (Interleukin-1) signaling pathway molecule contained in the specimen as an indicator. The present inventors have also found that by using the level of the IL-1 signaling pathway molecule as an indicator, a change in responsiveness to an immune checkpoint inhibitor after starting treatment (including acquisition of therapeutic resistance and the like) can be predicted. The present inventors have also found that by using the level of the IL-1 signaling pathway molecule as an indicator, the prognosis of a cancer patient receiving treatment with an immune checkpoint inhibitor can be predicted. The present invention is based on these findings.

An object of the present invention is to provide a method for predicting responsiveness to an immune checkpoint inhibitor. Another object of the present invention is to provide a method for predicting prognosis of a subject suffering from cancer who has received treatment with an immune checkpoint inhibitor.

According to the present invention, the following inventions are provided.
[1] A method for predicting responsiveness to an immune checkpoint inhibitor, the method comprising predicting therapeutic responsiveness of a subject in need of treatment of cancer to an immune checkpoint inhibitor by using an amount or concentration of an IL-1 signaling pathway molecule in a biological sample of the subject as an indicator.
[2] The method according to [1], comprising a step of measuring the amount or concentration of the IL-1 signaling pathway molecule in the biological sample of the subject.
[3] The method according to [1] or [2], comprising a step of comparing the amount or concentration of the IL-1 signaling pathway molecule in the biological sample of the subject with a cutoff value.
[4] The method according to any one of [1] to [3], wherein the IL-1 signaling pathway molecule is one or two or more substances (IL-1 signaling pathway molecule (a)) selected from the group consisting of (l) IL-1RAP, (2) IL-1R2, (3) IL-1R1, (4) ST2 (IL-1RL1), and (5) IL-1Rrp2.
[5] The method according to [4], wherein the amount or concentration of the IL-1 signaling pathway molecule (a) in the biological sample of the subject before or after starting treatment with an immune checkpoint inhibitor being higher than a cutoff value indicates that the subject is responsive to the immune checkpoint inhibitor.
[6] The method according to any one of [1] to [3], wherein the IL-1 signaling pathway molecule is one or two or more substances (IL-1 signaling pathway molecule (b)) selected from the group consisting of (11) IL-1β, (12) IL-1α, (13) IL-1Ra, (14) IL-33, (15) IL-38, (16) IL-36α, (17) IL-363, (18) IL-36γ, and (19) IL-36Ra.
[7] The method according to [6], wherein the amount or concentration of the IL-1 signaling pathway molecule (b) in the biological sample of the subject before or after starting treatment with an immune checkpoint inhibitor being lower than a cutoff value indicates that the subject is responsive to the immune checkpoint inhibitor.
[8] The method according to any one of [1] to [3], wherein the IL-1 signaling pathway molecule is two or more substances selected from the group consisting of (1) IL-TRAP, (2) IL-1R2, (3) IL-1R1, (4) ST2 (IL-1RL1), (5) IL-1Rrp2, (11) IL-1β, (12) IL-1α, (13) IL-1Ra, (14) IL-33, (15) IL-38, (16) IL-36α, (17) IL-363, (18) IL-36γ, and (19) IL-36Ra.
[9] The method according to [8], wherein one composite value calculated from a measured value of the amount or concentration of two or more IL-1 signaling pathway molecules in the biological sample of the subject before or after starting treatment with an immune checkpoint inhibitor being higher or lower than a cutoff value indicates that the subject is responsive to the immune checkpoint inhibitor.
[10] The method according to any one of [1] to [9], wherein the biological sample is a blood sample.
[11] The method according to any one of [1] to [10], which is a biological sample analysis method for predicting responsiveness to an immune checkpoint inhibitor.
[12] A method for predicting prognosis of a subject suffering from cancer who has received treatment with an immune checkpoint inhibitor, the method comprising predicting the prognosis by using an amount or concentration of an IL-1 signaling pathway molecule in a biological sample of the subject as an indicator.
[13] The method according to [12], comprising a step of measuring the amount or concentration of the IL-1 signaling pathway molecule in the biological sample of the subject.
[14] The method according to [12] or [13], comprising a step of comparing the amount or concentration of the IL-1 signaling pathway molecule in the biological sample of the subject with a cutoff value.
[15] Use of an IL-1 signaling pathway molecule as a biomarker for predicting responsiveness to an immune checkpoint inhibitor or a biomarker for predicting prognosis of a subject suffering from cancer who has received treatment with an immune checkpoint inhibitor.
[16] A prediction kit of responsiveness to an immune checkpoint inhibitor or a prediction kit of prognosis of a subject suffering from cancer who has received treatment with an immune checkpoint inhibitor, comprising a means for quantifying an amount or concentration of an IL-1 signaling pathway molecule in a biological sample.
[17] A method for treating cancer in a subject predicted to be responsive to treatment with an immune checkpoint inhibitor, the method comprising selecting the subject by the method according to any one of [1] to [14], and subjecting the selected subject to treatment with the immune checkpoint inhibitor.
[18] A method for treating cancer in a subject undergoing treatment with an immune checkpoint inhibitor, the method comprising selecting a subject predicted to be non responsive to treatment with the immune checkpoint inhibitor by the method according to any one of [1] to [14] and subjecting the selected subject to treatment other than the treatment with the immune checkpoint inhibitor.

According to the present invention, there is provided a novel biomarker for predicting responsiveness to an immune checkpoint inhibitor. The present invention is advantageous in that it contributes to improvement of prediction accuracy of responsiveness to an immune checkpoint inhibitor and improvement of prognosis of a cancer patient.

### BRIEF DESCRIPTION OF THE DRAWINGS

FIG. 1 is a view showing temporal changes in serum proteins (IL-1RAF, Gelsolin, or α1 acid glycoprotein 1) in LLC tumor-bearing mice. Measured values were expressed as mean f standard deviation (n=6). *P<0.05, **P<0.01, ***P<0.001 v.s. control group

FIG. 2 is a view showing concentration variations of serum proteins (IL-1RAP, Gelsolin, oral acid glycoprotein 1) in various tumor-bearing mice. Measured values were expressed as mean±standard deviation (n=6). *P<0.05, ***P<0.001 v.s. control group, ##P<0.01, ###P<0.001 v.s. MC38

FIG. 3 is a view showing an IL-1RAP concentration in the course of treatment of a cancer patient. In all cases (response case n=16, non-response case n=34, FIG. 3A), lung cancer cases (response case n=7, non-response case n=14, FIG. 3B), and renal cancer cases (response case n=9, non-response case n=20, FIG. 3C), the IL-1RAP concentration was significantly higher in the response group than in the non-response group from before starting treatment, and decreased at a stage showing therapeutic resistance. **P<0.01, ***P<0.001

FIG. 4 is a view showing a Gelsolin concentration in the course of treatment of a cancer patient. In all cases (response case n=16, non-response case n=34, FIG. 4A), lung cancer cases (response case n=7, non-response case n=14, FIG. 4B), and renal cancer cases (response case n=9, non-response case n=20, FIG. 4C), no significant difference in Gelsolin concentration was observed between the response group and the non-response group.

FIG. 5 is a view showing an α1 acid glycoprotein 1 concentration in the course of treatment of a cancer patient. In all cases (response case n=16, non-response case n=34, FIG. 5A), lung cancer cases (response case n=7, non-response case n=14, FIG. 5B), and renal cancer cases (response case n=9, non-response case n=20, FIG. 5C), no significant difference in α1 acid glycoprotein 1 concentration was observed between the response group and the non-response group.

FIG. 6 is a view showing therapeutic responsiveness prediction for an immune checkpoint inhibitor-administered patient based on an IL-1RAP concentration before starting treatment using an ROC curve.

FIG. 7 is a view showing a progression-free survival rate evaluated using an IL-TRAP concentration before starting treatment in all cases of an immune checkpoint inhibitor-administered patient. ***P<0.001

FIG. 8A is a view showing an IL-1RAP concentration in the course of treatment of all cancer patient cases (lung cancer cases (response case n=7, non-response case n=14), renal cancer cases (response case n=9, non-response case n=20), the same applies hereinafter). The results obtained by performing a significant difference test (Welch's t-test) between the response group and the non-response group for each of the lung cancer cases and the renal cancer cases are shown in the table (the same applies hereinafter). FIG. 8B is a view showing therapeutic responsiveness prediction for an immune checkpoint inhibitor-administered patient based on an CL-1 RAP concentration before starting treatment using an ROC curve (vertical axis: true positive rate, horizontal axis: false positive rate, the same applies hereinafter). FIG. 8C is a view showing a progression-free survival rate evaluated using an IL-1RAP concentration before starting treatment in all cases of an immune checkpoint inhibitor-administered patient (vertical axis: progression-free survival rate (×100%), horizontal axis: passage (days), the same applies hereinafter).

FIG. 9 is a view showing a correlation between an IL-1RAP concentration and an IL-1R2 concentration in serum.

FIG. 10A is a view showing an IL-1R2 concentration in the course of treatment of a cancer patient. FIG. 10B is a view showing therapeutic responsiveness prediction for an immune checkpoint inhibitor-administered patient based on an IL-1R2 concentration before starting treatment using an ROC curve. FIG. 10C is a view showing a progression-free survival rate evaluated using an IL-1R2 concentration before starting treatment in all cases of an immune checkpoint inhibitor-administered patient.

FIG. 11A is a view showing a composite value of an IL-1RAP concentration and an IL-1R2 concentration in the course of treatment of a cancer patient. FIG. 11B is a view showing therapeutic responsiveness prediction for an immune checkpoint inhibitor-administered patient based on a composite value of an IL-1RAP concentration and an IL-1R2 concentration before starting treatment using an ROC curve. FIG. 11C is a view showing a progression-free survival rate evaluated using a composite value of an IL-1 RAP concentration and an IL-1R2 concentration before starting treatment in all cases of an immune checkpoint inhibitor-administered patient.

FIG. 12 is a view showing a correlation between an IL-1β (Interleukin-1 beta) concentration in serum and changes in the concentrations of IL-1RAP and IL-1R2 (separating plane: 0.0824×IL1RAP+1.2269×IL1-R2−2.7216×IL-13=19.0478).

FIG. 13A is a view showing an IL-1β concentration in the course of treatment of a cancer patient. FIG. 13B is a view showing therapeutic responsiveness prediction for an immune checkpoint inhibitor-administered patient based on an IL-1β concentration before starting treatment using an ROC curve. FIG. 13C is a view showing a progression-free survival rate evaluated using an IL-1β concentration before starting treatment in all cases of an immune checkpoint inhibitor-administered patient.

FIG. 14A is a view showing a composite value of an IL-1β concentration and an IL-1RAP concentration in the course of treatment of a cancer patient. FIG. 14B is a view showing therapeutic responsiveness prediction for an immune checkpoint inhibitor-administered patient based on a composite value of an IL-1β concentration and an IL-1RAP concentration before starting treatment using an ROC curve. FIG. 14C is a view showing a progression-free survival rate evaluated using an IL-1β concentration and an IL-1RAP concentration before starting treatment in all cases of an immune checkpoint inhibitor-administered patient.

FIG. 15A is a view showing a composite value of an IL-1β concentration and an IL-1R2 concentration in the course of treatment of a cancer patient. FIG. 15B is a view showing therapeutic responsiveness prediction for an immune checkpoint inhibitor-administered patient based on a composite value of an IL-1β concentration and an IL-1R2 concentration before starting treatment using an ROC curve. FIG. 15C is a view showing a progression-free survival rate evaluated using an IL-1β concentration and an IL-1R2 concentration before starting treatment in all cases of an immune checkpoint inhibitor-administered patient.

FIG. 16A is a view showing a composite value of an IL-1RAP concentration, an IL-1R2 concentration, and an IL-1β concentration in the course of treatment of a cancer patient. FIG. 16B is a view showing therapeutic responsiveness prediction for an immune checkpoint inhibitor-administered patient based on a composite value of an IL-1 RAP concentration, an IL-1R2 concentration, and an IL-1β concentration before starting treatment using an ROC curve. FIG. 16C is a view showing a progression-free survival rate evaluated using an IL-1RAP concentration, and IL-1R2, and an IL-1β concentration before starting treatment in all cases of an immune checkpoint inhibitor-administered patient.

FIG. 17A is a view showing an IL-1β concentration in the course of treatment of a cancer patient. FIG. 17B is a view showing therapeutic responsiveness prediction for an immune checkpoint inhibitor-administered patient based on a composite value of an IL-1R1 concentration before starting treatment using an ROC curve. FIG. 17C is a view showing a progression-free survival rate evaluated using an IL-1R1 concentration before starting treatment in all cases of an immune checkpoint inhibitor-administered patient.

FIG. 18A is a view showing a composite value of an IL-1R1 concentration and an IL-1RAP concentration in the course of treatment of a cancer patient. FIG. 18B is a view showing therapeutic responsiveness prediction for an immune checkpoint inhibitor-administered patient based on a composite value of an IL-1R1 concentration and an IL-1RAP concentration before starting treatment using an ROC curve. FIG. 18C is a view showing a progression-free survival rate evaluated using a composite value of an IL-1R1 concentration and an IL-1RAP concentration before starting treatment in all cases of an immune checkpoint inhibitor-administered patient.

FIG. 19A is a view showing a composite value of an IL-1R1 concentration and an IL-1R2 concentration in the course of treatment of a cancer patient. FIG. 19B is a view showing therapeutic responsiveness prediction for an immune checkpoint inhibitor-administered patient based on a composite value of an IL-1R1 concentration and an IL-1 R2 concentration before starting treatment using an ROC curve. FIG. 19C is a view showing a progression-free survival rate evaluated using a composite value of an IL-1R1 concentration and an IL-1R2 concentration before starting treatment in all cases of an immune checkpoint inhibitor-administered patient.

FIG. 20A is a view showing a composite value of an IL-1R1 concentration and an IL-1β concentration in the course of treatment of a cancer patient. FIG. 20B is a view showing therapeutic responsiveness prediction for an immune checkpoint inhibitor-administered patient based on a composite value of an IL-1R1 concentration and an IL-1β concentration before starting treatment using an ROC curve. FIG. 20C is a view showing a progression-free survival rate evaluated using a composite value of an IL-1R1 concentration and an IL-1p concentration before starting treatment in all cases of an immune checkpoint inhibitor-administered patient.

FIG. 21A is a view showing a composite value of an IL-1R1 concentration, an IL-1RAP concentration, and an IL-1β concentration in the course of treatment of a cancer patient. FIG. 21B is a view showing therapeutic responsiveness prediction for an immune checkpoint inhibitor-administered patient based on a composite value of an IL-1R1 concentration, an IL-1RAP concentration, and an IL-1β concentration before starting treatment using an ROC curve.

FIG. 21C is a view showing a progression-free survival rate evaluated using a composite value of an IL-1R1 concentration, an IL-1RAP concentration, and an IL-1β concentration before starting treatment in all cases of an immune checkpoint inhibitor-administered patient.

FIG. 22A is a view showing a composite value of an IL-1R1 concentration, an IL-1R2 concentration, and an IL-1β concentration in the course of treatment of a cancer patient. FIG. 22B is a view showing therapeutic responsiveness prediction of an immune checkpoint inhibitor-administered patient based on a composite value of an IL-1R1 concentration, an IL-1R2 concentration, and an IL-1β concentration before starting treatment using an ROC curve.

FIG. 22C is a view showing a progression-free survival rate evaluated using a composite value of an IL-1R1 concentration, an IL-1R2 concentration, and an IL-1β concentration before starting treatment in all cases of an immune checkpoint inhibitor-administered patient.

FIG. 23A is a view showing a composite value of an IL-1R1 concentration, an IL-1R2 concentration, and an IL-TRAP concentration in the course of treatment of a cancer patient.

FIG. 23B is a view showing therapeutic responsiveness prediction for an immune checkpoint inhibitor-administered patient based on a composite value of an IL-1R1 concentration, an IL-1R2 concentration, and an IL-TRAP concentration before starting treatment using an ROC curve. FIG. 23C is a view showing a progression-free survival rate evaluated using a composite value of an IL-1R1 concentration, an IL-1 R2 concentration, and an IL-1 RAP concentration before starting treatment in all cases of an immune checkpoint inhibitor-administered patient.

FIG. 24A is a view showing a composite value of an IL-1R1 concentration, an IL-1R2 concentration, an IL-TRAP concentration, and an IL-1β concentration in the course of treatment of a cancer patient. FIG. 24B is a view showing therapeutic responsiveness prediction for an immune checkpoint inhibitor-administered patient based on a composite value of an IL-1R1 concentration, an IL-1R2 concentration, an IL-1RAP concentration, and an IL-1β concentration before starting treatment using an ROC curve. FIG. 24C is a view showing a progression-free survival rate evaluated using a composite value of an IL-1R1 concentration, an IL-1R2 concentration, an IL-1 RAP concentration, and an IL-1β concentration before starting treatment in all cases of an immune checkpoint inhibitor-administered patient.

### DETAILED DESCRIPTION OF THE INVENTION

### Definitions

In the present invention, the term “cancer” means cancer to be treated with an immune checkpoint inhibitor. Examples of the cancer to be treated with an immune checkpoint inhibitor include, but are not limited to, malignant melanoma, non-small cell lung cancer, small cell lung cancer, malignant pleural mesothelioma, hepatocellular carcinoma, gastric cancer, head and neck cancer, esophageal cancer, renal cell carcinoma, urothelial cancer, breast cancer, uterine body cancer, solid cancer having high frequency microsatellite instability (MSI-High), and Hodgkin's lymphoma.

In the present invention, the term “subject” includes mammals including humans suffering from cancer, and is preferably humans suffering from cancer.

In the present invention, the term “biological sample” means a sample separated from a living body, and represents, for example, a body fluid such as blood, preferably serum or plasma. A method for collecting the biological sample may be invasive, minimally invasive, or noninvasive, and is advantageous in that the blood sample can be collected minimally invasive when the biological sample is a blood sample.

In the present invention, an IL-1 signaling pathway molecule means a molecule involved in a signaling pathway regulated by a cytokine (IL-1 cytokine) belonging to the IL-1 cytokine family. Examples of such a molecule include an IL-1 cytokine and a receptor of the IL-1 cytokine. Examples of the IL-1 cytokine include IL-1β, IL-1α, IL-1Ra, IL-33, IL-38, IL-36α, 11-3613, IL-36γ, and IL-36Ra. Examples of the receptor of the IL-1 cytokine include IL-1RAP, IL-1R2, IL-1R1, ST2 (IL-1RL1), and IL-1Rrp2.

In the present invention, the IL-1 signaling pathway molecule can include at least one or two or more substances selected from the group consisting of:
(1) IL-1RAP,
(2) IL-1 R2,
(3) IL-1R1,
(4) ST2 (IL-1 RL1), and
(5) IL-1Rrp2.

In the present specification, one or two or more substances selected from the group consisting of the above (1) to (5) may be referred to as “IL-1 signaling pathway molecule (a) of the present invention” or “IL-1 signaling pathway molecule (a)”. The IL-1 signaling pathway molecule (a) of the present invention can be preferably one, two, or three substances selected from the group consisting of the above (1) to (3).

In the present invention, the IL-1 signaling pathway molecule can also include at least one or two or more substances selected from the group consisting of.
(11) IL-1β,
(12) IL-1α,
(13) IL-1Ra,
(14) IL-33,
(15) IL-38,
(16) IL-36α,
(17) IL-36β,
(18) IL-36γ, and
(19) IL-36Ra.

In the present specification, one or two or more substances selected from the group consisting of the above (11) to (19) may be referred to as “IL-1 signaling pathway molecule (b) of the present invention” or “IL-1 signaling pathway molecule (b)”. The IL-1 signaling pathway molecule (b) of the present invention can be preferably one, two, or three substances selected from the group consisting of the above (11) to (13). Without being bound by the following theory, it is considered that the cytokines (11) to (19) can bind to at least one of the receptors (1) to (3), respectively, and thus, the cytokines exhibit behaviors correlated with the receptors (1) to (3) with respect to responsiveness to an immune checkpoint inhibitor. That is, the IL-1 signaling pathway molecule of the present invention can also be a cytokine capable of binding to at least any one of the receptors (1) to (3).

In the present invention, the IL-1 signaling pathway molecule (a) of the present invention and the IL-1 signaling pathway molecule (b) of the present invention may be collectively referred to as the IL-1 signaling pathway molecule of the present invention. In the present invention, IL-1 signaling pathway molecule (a) and the IL-1 signaling pathway molecule (b) may be collectively referred to as the IL-1 signaling pathway molecule. The IL-1 signaling pathway molecule of the present invention can be one or two or more substances selected from the group consisting of the above (1) to (5) and (ii) to (19), and is preferably one, two, three, or four substances selected from the group consisting of the above (1) to (3) and (11) to (13) or the above (1) to (3) and (11), and more preferably two, three, or four substances selected from the group consisting of the above (1) to (3) and (11) from the viewpoint of prediction accuracy.

In the present invention, the “immune checkpoint inhibitor” means a substance that inhibits the function of an immune checkpoint molecule. The immune checkpoint molecule is a molecular group that suppresses an immune response to self in order to maintain immune homeostasis and suppresses an excessive immune reaction. Examples of the immune checkpoint inhibitor include, but are not limited to, anti-PD-L1 antibodies, anti-PD-1 antibodies, and anti-CTLA-4 antibodies. Examples of the anti-PD-1 antibodies include nivolumab, pembrolizumab, cemiplimab, and PDR001. Examples of the anti-PD-L1 antibodies include avelumab, atezolizumab, and durvalumab. Examples of the anti-CTLA-4 antibodies include ipilimumab and tremelimumab.

In the present invention, “responsiveness to an immune checkpoint inhibitor” means whether or not the cancer of the subject is improved by administration of an immune checkpoint inhibitor. The improvement of cancer means that the cancer regresses or the cancer does not increase, and includes that the size of the cancer is unchanged. It can be said that “cancer is improved” is “responsive” and “cancer is not improved” is “non-responsive”. It can be said that a case where a subject who was responsive to an immune checkpoint inhibitor at the stage of starting treatment with an immune checkpoint inhibitor changed to be therapeutically resistant to the immune checkpoint inhibitor during the period of continuing the treatment with an immune checkpoint inhibitor and the treatment with an immune checkpoint inhibitor is invalidated is “non-responsiveness to the immune checkpoint inhibitor after starting treatment”.

### <<Method for Predicting Therapeutic Responsiveness>>

According to the present invention, there is provided a method for predicting responsiveness to an immune checkpoint inhibitor. According to the method for predicting responsiveness of the present invention, responsiveness can be predicted using an amount or concentration of an IL-1 signaling pathway molecule in a biological sample of a test subject as an indicator. That is, the method for predicting responsiveness of the present invention is characterized by associating the amount or concentration of the IL-1 signaling pathway molecule in the biological sample with responsiveness to an immune checkpoint inhibitor in the test subject. Since the method for predicting responsiveness of the present invention has an aspect of determining (deciding) responsiveness using the amount or concentration of the IL-1 signaling pathway molecule in the biological sample of the test subject as an indicator, the method for predicting responsiveness of the present invention can be rephrased as a method for determining responsiveness.

In the method for predicting responsiveness of the present invention, (A) a step of measuring an amount or concentration of an IL-1 signaling pathway molecule of the present invention in a biological sample of a test subject can be performed. The step (A) can be (A-1) a step of measuring an amount or concentration of an IL-1 signaling pathway molecule of the present invention in a biological sample of a test subject before starting treatment with an immune checkpoint inhibitor, or (A-2) a step of measuring an amount or concentration of an IL-1 signaling pathway molecule of the present invention in a biological sample of a test subject after starting treatment with an immune checkpoint inhibitor.

The measurement of the amount and concentration of the IL-1 signaling pathway molecule of the present invention can be performed by selecting a known method depending on the properties of the biological sample and the substance. The measurement of the amount and concentration of the IL-1 signaling pathway molecule of the present invention can be performed by a known method, and for example, a measurement method using a substance that specifically binds to the IL-1 signaling pathway molecule can be used. Typical examples of the substance that specifically binds to the IL-1 signaling pathway molecule include antibodies, aptamers (for example, nucleic acid aptamers or peptide aptamers), and drugs. When an antibody is used as the substance that specifically binds to the IL-1 signaling pathway molecule, the amount or concentration of the IL-1 signaling pathway molecule can be measured, for example, by immunoassay. The immunoassay is an analysis method using a detectably labeled anti-IL-1 signaling pathway molecule antibody, a detectably labeled antibody (secondary antibody) to an anti-IL-1 signaling pathway molecule antibody, or the like. The method for labeling an antibody is classified into enzyme immunoassay (EIA or ELISA), radioimmunoassay (RIA), fluorescence immunoassay (FIA), fluorescence polarization immunoassay (FPIA), chemiluminescence immunoassay (CLIA), and the like, and the IL-1 signaling pathway molecule can be detected or quantified by an absorption method, a fluorescence method, a polarized fluorescence method, a chemiluminescence method, a bioluminescence method, an electroconductivity detection method, an electrochemical detection method, an enzyme method, a method using a radioactive substance, or a method combining these methods.

When the IL-1 signaling pathway molecule of the present invention is measured, the measurement can also be performed by an analysis system to which a mass spectrometer is connected.

In the method for predicting responsiveness of the present invention, responsiveness can be predicted based on the measurement result of the IL-1 signaling pathway molecule in the biological sample of the test subject. That is, the method for predicting responsiveness of the present invention can include (B) a step of predicting or determining responsiveness to an immune checkpoint inhibitor for a test subject from which a biological sample is collected, using the amount or concentration of the IL-1 signaling pathway molecule as an indicator. The step (B) may further include a step of comparing the amount or concentration of the IL-1 signaling pathway molecule in the biological sample of the test subject with a cutoff value.

When a measurement target is the IL-1 signaling pathway molecule (a), an amount or concentration of the IL-1 signaling pathway molecule (a) in the biological sample of the test subject before or after starting treatment with an immune checkpoint inhibitor being higher than a cutoff value indicates that the subject is responsive to the immune checkpoint inhibitor. When a measurement target is the IL-1 signaling pathway molecule (a), an amount or concentration of the IL-1 signaling pathway molecule (a) in the biological sample of the test subject before or after starting treatment with an immune checkpoint inhibitor being lower than a cutoff value indicates that the subject is non-responsive to the immune checkpoint inhibitor.

That is, when a measurement target is the IL-1 signaling pathway molecule (a), the step (B) can be performed by (B-a-1) a step of comparing the amount or concentration of the IL-1 signaling pathway molecule (a) in the biological sample of the test subject with a predetermined cutoff value, and (B-a-2) a step of predicting or determining that the test subject is responsive to the immune checkpoint inhibitor when the amount or concentration of the IL-1 signaling pathway molecule (a) in the biological sample of the test subject is equal to or higher than a cutoff value or is higher than a cutoff value. In the step (B-a-2), it can also be predicted or determined that the test subject is non-responsive to the immune checkpoint inhibitor when the amount or concentration of the IL-1 signaling pathway molecule (a) in the biological sample of the test subject is equal to or lower than a cutoff value or is lower than a cutoff value.

When a measurement target is the IL-1 signaling pathway molecule (b), an amount or concentration of the IL-1 signaling pathway molecule (b) in the biological sample of the subject before or after starting treatment with an immune checkpoint inhibitor being lower than a cutoff value indicates that the subject is responsive to the immune checkpoint inhibitor. When a measurement target is the IL-1 signaling pathway molecule (b), an amount or concentration of the IL-1 signaling pathway molecule (b) in the biological sample of the subject before or after starting treatment with an immune checkpoint inhibitor being higher than a cutoff value indicates that the subject is non-responsive to the immune checkpoint inhibitor

That is, when a measurement target is the IL-1 signaling pathway molecule (b), the step (B) can be performed by (B-b-1) a step of comparing the amount or concentration of the IL-1 signaling pathway molecule (b) in the biological sample of the test subject with a predetermined cutoff value, and (B-b-2) a step of predicting or determining that the test subject is responsive to the immune checkpoint inhibitor when the amount or concentration of the IL-1 signaling pathway molecule (b) in the biological sample of the test subject is equal to or lower than a cutoff value or is lower than a cutoff value. In the step (B-b-2), it can also be predicted or determined that the test subject is non-responsive to the immune checkpoint inhibitor when the amount or concentration of the IL-1 signaling pathway molecule (b) in the biological sample of the test subject is equal to or higher than a cutoff value or is higher than a cutoff value.

By performing the step (B) using the amount or concentration of the IL-1 signaling pathway molecule of the present invention measured in the step (A-1) as an indicator, responsiveness to an immune checkpoint inhibitor can be predicted before starting treatment with an immune checkpoint inhibitor for a test subject from which a biological sample is collected. In this case, in the step (B-2), when the test subject is predicted to be responsive to an immune checkpoint inhibitor, it is recommended that the test subject receives treatment with an immune checkpoint inhibitor. On the other hand, in the step (B-2), when the test subject is predicted to be non-responsive to an immune checkpoint inhibitor, it is recommended that the test subject receives treatment other than the treatment with an immune checkpoint inhibitor.

By performing the step (B) using the amount or concentration of the IL-1 signaling pathway molecule of the present invention measured in the step (A-2) as an indicator, responsiveness to an immune checkpoint inhibitor can be predicted after starting treatment with an immune checkpoint inhibitor for a test subject from which a biological sample is collected. In this case, in the step (B-2), when the test subject is predicted to be responsive to an immune checkpoint inhibitor, it is recommended that the test subject continues the treatment with an immune checkpoint inhibitor. On the other hand, in the step (B-2), when the test subject is predicted to be non-responsive to an immune checkpoint inhibitor (that is, the treatment is invalidated due to therapeutic resistance), it is recommended that the test subject terminates the treatment other than the treatment with an immune checkpoint inhibitor.

When prediction is performed by combining two or more kinds of IL-1 signaling pathway molecules of the present invention in the method for predicting responsiveness of the present invention, responsiveness to an immune checkpoint inhibitor can be predicted more accurately as compared with a case where prediction is performed using the IL-1 signaling pathway molecule alone.

When prediction is performed by combining two or more kinds of IL-1 signaling pathway molecules of the present invention in the method for predicting responsiveness of the present invention, the step (A) and the step (B) can be performed for each IL-1 signaling pathway molecule. In this case, therapeutic responsiveness can be predicted by combining the prediction results of the therapeutic responsiveness shown based on the respective IL-1 signaling pathway molecules. For example, when responsiveness is predicted for both two kinds of IL-1 signaling pathway molecules of the present invention, possibility of responsiveness is strongly suggested as compared to the results for each CL-1 signaling pathway molecule alone, and when non-responsiveness is predicted for both two kinds of IL-1 signaling pathway molecules of the present invention, possibility of non-responsiveness is strongly suggested as compared to the results for each IL-1 signaling pathway molecule alone. When prediction is performed by combining two or more kinds of IL-1 signaling pathway molecules of the present invention in the method for predicting responsiveness of the present invention, as described below, a single value (composite value) can be calculated using the total value, average value, ratio, or the like of the measured values of the amount or concentration of a plurality of kinds of IL-1 signaling pathway molecules, or after weighting each measured value of the amount or concentration of a plurality of kinds of IL-1 signaling pathway molecules, a single value (composite value) can be calculated using the total value, average value, ratio, or the like thereof.

When prediction is performed by combining two or more kinds of IL-1 signaling pathway molecules of the present invention in the method for predicting responsiveness of the present invention, two, three, or four kinds of cytokines selected from the group consisting of (1) IL-1RAP, (2) IL-1R2, (3) IL-1R1, and (11) IL-1β can be used as an indicator.

In the method for predicting responsiveness of the present invention, a known biomarker can be used as an indicator in combination with an IL-1 signaling pathway molecule. When prediction is performed by combining a known biomarker in addition to the IL-1 signaling pathway molecule in the method for predicting responsiveness of the present invention, responsiveness to an immune checkpoint inhibitor can be predicted more accurately as compared with a case where prediction is performed using the IL-1 signaling pathway molecule alone.

In the present invention, the cutoff value can be calculated and determined from the measured value of the amount or concentration of the IL-1 signaling pathway molecule of the present invention in a sample at a predetermined time point in a group responsive to an immune checkpoint inhibitor (response group) among the patient group to which the immune checkpoint inhibitor has been administered. Such a subject maybe a subject having a disease other than cancer. In the present invention, the cutoff value can also be calculated and determined from the measured value of the amount or concentration of the metabolite of the present invention in a sample at a predetermined time point in a group non-responsive to an immune checkpoint inhibitor (non-response group) among the patient group to which the immune checkpoint inhibitor has been administered. In the method for determining the cutoff value, the average value, median value, percentile value, maximum value, or minimum value of the measured values of the response group or the non-response group can be used. The percentile value can be any value, for example, 5, 10, 15, 20, 25, 30, 40, 50, 60, 70, 75, 80, 85, 90, or 95. The number of examples of the response subject and the non-response subject when the cutoff value is calculated is preferably plural, and can be, for example, 2 or more, 5 or more, 10 or more, 20 or more, 50 or more, or 100 or more.

In the present invention, the cutoff value can also be calculated based on the measured value of the amount or concentration of the IL-1 signaling pathway molecule of the present invention in a sample at a predetermined time point in a group responsive to an immune checkpoint inhibitor (response group) among the patient group to which the immune checkpoint inhibitor has been administered and the measured value of the amount or concentration of the IL-1 signaling pathway molecule of the present invention in a sample at a predetermined time point in a group non-responsive to an immune checkpoint inhibitor (non-response group) among the patient group to which the immune checkpoint inhibitor has been administered. For example, the cutoff value can be set by measuring the amount or concentration of the IL-1 signaling pathway molecule of the present invention in a biological sample for the response group and the non-response group, and performing statistical analysis such as receiver operating characteristic curve (ROC) analysis using the obtained measured value. The preparation of the ROC curve and the setting of the cutoff value based on the ROC curve are well known, and those skilled in the art can set the cutoff value from the viewpoint of sensitivity and specificity.

In the method for predicting responsiveness of the present invention, depending on the aspect, the biological sample can be a biological sample at a predetermined time point. For example, in an aspect in which responsiveness to an immune checkpoint inhibitor of a test subject is predicted before starting treatment with an immune checkpoint inhibitor, a biological sample of the test subject and a biological sample used for calculation of a cutoff value can be a biological sample before starting treatment with a checkpoint inhibitor. In an aspect in which responsiveness to an immune checkpoint inhibitor of a test subject is predicted after starting treatment with an immune checkpoint inhibitor, a biological sample of the test subject and a biological sample used for calculation of a cutoff value can be a biological sample after starting treatment with a checkpoint inhibitor. The biological sample after starting treatment with a checkpoint inhibitor may be, for example, a biological sample after one week, two weeks, three weeks, one month, two months, three months, or four months from starting treatment, or can be appropriately set according to the number of courses of administration, such as one course, two courses, three courses, or four courses after starting treatment, but is not limited thereto. The term “course” means one group of the administration period and the rest period of the immune checkpoint inhibitor, and may be referred to as “cycle” or “Kur”.

In the method for predicting responsiveness of the present invention, when another substance (for example, a known biomarker) is used as an indicator in addition to the IL-1 signaling pathway molecule of the present invention, the cutoff value of the another substance can be calculated and determined according to the description of the cutoff value of the IL-1 signaling pathway molecule.

In the step (B) of the method for predicting responsiveness of the present invention, for example, when the amount or concentration of the IL-1 signaling pathway molecule (a) in the biological sample of the test subject is higher than the average value of the amount or concentration of the IL-1 signaling pathway molecule in the non-response group or is about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2.0 times or more, about 2.1 times or more, about 2.2 times or more, about 2.3 times or more, about 2.4 times or more, about 2.5 times or more, or about 3 times or more as compared to the average value, it can be predicted or determined that the test subject is responsive to the immune checkpoint inhibitor.

In the step (B) of the method for predicting responsiveness of the present invention, for example, when the amount or concentration of the IL-1 signaling pathway molecule (b) in the biological sample of the test subject is lower than the average value of the amount or concentration of the IL-1 signaling pathway molecule in the non-response group or is about 0.9 times or less, about 0.85 times or less, about 0.8 times or less, about 0.75 times or less, about 0.7 times or less, about 0.65 times or less, about 0.6 times or less, about 0.55 times or less, about 0.5 times or less, about 0.45 times or less, about 0.4 times or less, or about 0.35 times or less as compared to the average value, it can also be predicted or determined that the test subject is responsive to the immune checkpoint inhibitor.

In the present invention, the prediction accuracy can be improved by using a combination of a plurality of kinds of IL-1 signaling pathway molecules of the present invention. In the present invention, the prediction accuracy can be further improved by using the IL-1 signaling pathway molecule of the present invention in combination with another substance (for example, a known biomarker). Here, the improvement in the prediction accuracy means that the area under the curve (AUC) of the ROC curve is improved in the case of using ROC analysis.

In the present invention, when a plurality of kinds of IL-1 signaling pathway molecules of the present invention are combined and used as an indicator or when the IL-1 signaling pathway molecule of the present invention is used as an indicator in combination with another substance (for example, a known biomarker), one cutoff value can also be set for the measured value of the amount or concentration of the plurality of kinds of IL-1 signaling pathway molecules as an indicator or the measured value of the amount or concentration of one or a plurality of kinds of IL-1 signaling pathway molecules and the another substance as an indicator. For example, the cutoff value can be calculated using the total value, average value, ratio, or the like of the measured values of the amount or concentration of a plurality of kinds of IL-1 signaling pathway molecule instead of the measured value of the amount or concentration of one kind of IL-1 signaling pathway molecules, or after weighting each measured value of the amount or concentration of a plurality of kinds of IL-1 signaling pathway molecules, the total value, average value, ratio, or the like thereof is calculated, and then the cutoff value can be calculated using the calculated value. When the cutoff value calculated in this way is used in the present invention, it is possible to perform prediction or determination by processing the measured value of the amount or concentration of a plurality of kinds of IL-1 signaling pathway molecules in a biological sample of a test subject in the same manner as in the method for calculating a cutoff value and comparing one obtained numerical value (composite value) with a predetermined cutoff value.

The method of weighting each measured value of the amount or concentration of a plurality of kinds of IL-1 signaling pathway molecules and then calculating the total value, average value, ratio, or the like thereof is known, and a coefficient for each signaling pathway molecule can be calculated according to linear discriminant analysis. Numerical analysis software for performing linear discriminant analysis is available, and for example, Matlab (MathWorks) can be used.

According to the method for predicting responsiveness of the present invention, responsiveness to an immune checkpoint inhibitor can be predicted for a test subject. Therefore, the method for predicting responsiveness of the present invention can be used supplementarily for treatment with an immune checkpoint inhibitor or diagnosis of efficacy of an immune checkpoint inhibitor, and whether or not a test subject is responsive to treatment with an immune checkpoint inhibitor can be finally determined by a medical doctor in combination with other findings in some cases. For example, for a test subject predicted to be responsive or non-responsive to an immune checkpoint inhibitor by the method for predicting responsiveness of the present invention, a medical doctor can determine whether the test subject is responsive or non-responsive to an immune checkpoint inhibitor while referring to other findings, and furthermore, can determine whether or not to continue treatment with an immune checkpoint inhibitor or the timing of switching to another agent. In particular, in the present invention, after starting treatment with an immune checkpoint inhibitor, the amount or concentration of the IL-1 signaling pathway molecule in a biological sample periodically obtained from a patient is measured, and the timing of switching the treatment method can be determined using the decrease or increase in the amount or concentration of the molecule as an indicator. That is, the method for predicting responsiveness of the present invention can be rephrased as a method for supplementing treatment with an immune checkpoint inhibitor or diagnosis of efficacy of an immune checkpoint inhibitor, or a method for assisting treatment with an immune checkpoint inhibitor or diagnosis of efficacy of an immune checkpoint inhibitor. The method for predicting responsiveness of the present invention leads to application of a drug to a cancer patient for which a therapeutic effect by an immune checkpoint inhibitor can be expected, and thus the present invention also contributes to reduction of medical costs and improvement of patient QOL.

According to the method for predicting responsiveness of the present invention, it is possible to analyze a biological sample collected from a test subject and quantitatively predict responsiveness to an immune checkpoint inhibitor. That is, the method for predicting responsiveness of the present invention is advantageous in that responsiveness to an immune checkpoint inhibitor can be easily and accurately predicted while reducing a burden on a patient. Therefore, the method for predicting responsiveness of the present invention can be rephrased as a method for analyzing a biological sample (preferably, a method for analyzing a blood sample) to predict responsiveness to an immune checkpoint inhibitor, or a method for monitoring or evaluating responsiveness to an immune checkpoint inhibitor.

### <<Method for Predicting Prognosis>>

According to a second aspect of the present invention, there is provided a method for predicting prognosis of a subject suffering from cancer who has received treatment with an immune checkpoint inhibitor. According to the method for predicting prognosis of the present invention, prognosis of a subject suffering from cancer who has received treatment with an immune checkpoint inhibitor can be predicted using an amount or concentration of an IL-1 signaling pathway molecule in a biological sample of a test subject as an indicator. That is, the method for predicting prognosis of the present invention is characterized by associating the amount or concentration of the IL-1 signaling pathway molecule in the biological sample with prognosis of a subject suffering from cancer who has received treatment with an immune checkpoint inhibitor.

In the method for predicting prognosis of the present invention, as in the method for predicting responsiveness of the present invention, (C) a step of measuring an amount or concentration of an IL-1 signaling pathway molecule of the present invention in a biological sample of a test subject before starting treatment with an immune checkpoint inhibitor can be performed. The measurement of the amount or concentration of the IL-1 signaling pathway molecule can be performed in the same manner as in the method for predicting responsiveness of the present invention.

In the method for predicting prognosis of the present invention, prognosis of a subject suffering from cancer who has received treatment with an immune checkpoint inhibitor can be predicted based on the measurement result of the IL-1 signaling pathway molecule in the biological sample of the test subject. That is, the method for predicting prognosis of the present invention can include (D) a step of predicting a possibility of prolongation of prognosis by an immune checkpoint inhibitor for a test subject from which a biological sample is collected, using the amount or concentration of the IL-1 signaling pathway molecule as an indicator. The step (D) may further include a step of comparing the amount or concentration of the IL-1 signaling pathway molecule in the biological sample of the test subject with a cutoff value. The prolongation of prognosis is used in the sense of including prolongation of progression-free survival time after starting treatment with an immune checkpoint inhibitor.

When a measurement target is the IL-1 signaling pathway molecule (a), an amount or concentration of the IL-1 signaling pathway molecule (a) in the biological sample of the test subject being higher than a cutoff value indicates that the subject has a possibility of prolongation of prognosis by the immune checkpoint inhibitor. That is, when a measurement target is the IL-1 signaling pathway molecule (a), the step (D) can be performed by (D-a-1) a step of comparing the amount or concentration of the IL-1 signaling pathway molecule (a) in the biological sample of the test subject with a predetermined cutoff value, and (D-a-2) a step of predicting or determining that there is a possibility of prolongation of prognosis by the immune checkpoint inhibitor when the amount or concentration of the IL-1 signaling pathway molecule (a) in the biological sample of the test subject is equal to or higher than a cutoff value or is higher than a cutoff value.

In the step (D-a-2), it can also be predicted or determined that a possibility of prolongation of prognosis by the immune checkpoint inhibitor is low when the amount or concentration of the IL-1 signaling pathway molecule (a) in the biological sample of the test subject is equal to or lower than a cutoff value or is lower than a cutoff value.

When a measurement target is the IL-1 signaling pathway molecule (b), an amount or concentration of the IL-1 signaling pathway molecule (b) in the biological sample of the test subject being lower than a cutoff value indicates that the subject has a possibility of prolongation of prognosis by the immune checkpoint inhibitor. That is, when a measurement target is the IL-1 signaling pathway molecule (b), the step (D) can be performed by (D-b-1) a step of comparing the amount or concentration of the IL-1 signaling pathway molecule (b) in the biological sample of the test subject with a predetermined cutoff value, and (D-b-2) a step of predicting or determining that there is a possibility of prolongation of prognosis by the immune checkpoint inhibitor when the amount or concentration of the IL-1 signaling pathway molecule (b) in the biological sample of the test subject is equal to or lower than a cutoff value or is lower than a cutoff value.

In the step (D-b-2), it can also be predicted or determined that a possibility of prolongation of prognosis by the immune checkpoint inhibitor is low when the amount or concentration of the IL-1 signaling pathway molecule (b) in the biological sample of the test subject is equal to or higher than a cutoff value or is higher than a cutoff value.

By performing the step (D) using the amount or concentration of the IL-1 signaling pathway molecule of the present invention measured in the step (C) as an indicator, a possibility of prolongation of prognosis by the immune checkpoint inhibitor can be predicted before starting treatment with an immune checkpoint inhibitor for a test subject from which a biological sample is collected. In this case, in the step (D), when it is predicted that there is a possibility of prolongation of prognosis by the immune checkpoint inhibitor, it is recommended that the test subject receives treatment with an immune checkpoint inhibitor. On the other hand, in the step (D), when the possibility of prolongation of prognosis by the immune checkpoint inhibitor is predicted to be low, it is recommended that the test subject receives treatment other than the treatment with an immune checkpoint inhibitor.

As in the method for predicting responsiveness of the present invention, the method for predicting prognosis of the present invention can be carried out by combining two or more kinds of IL-1 signaling pathway molecules of the present invention, and can also be carried out by combining a known biomarker with the IL-1 signaling pathway molecule.

The cutoff value in the method for predicting prognosis of the present invention can be determined in the same manner as in the method for predicting responsiveness of the present invention.

According to the method for predicting prognosis of the present invention, prognosis of a subject suffering from cancer who has received treatment with an immune checkpoint inhibitor can be predicted. Therefore, the method for predicting prognosis of the present invention can be used supplementarily for the diagnosis of the prognosis of a subject suffering from cancer who has received treatment with an immune checkpoint inhibitor, and the prognosis of the subject can be finally determined by a medical doctor in combination with other findings in some cases. For example, for a test subject predicted to have a possibility or low possibility of prolongation of prognosis by the immune checkpoint inhibitor by the method for predicting prognosis of the present invention, a medical doctor can determine whether there is a possibility or low possibility of prolongation of prognosis by the immune checkpoint inhibitor while referring to other findings, and furthermore, can determine whether or not treatment with an immune checkpoint inhibitor is appropriate or whether or not treatment with another agent is appropriate. That is, the method for predicting prognosis of the present invention can be rephrased as a method for supplementing prediction of prognosis of a subject suffering from cancer who has received treatment with an immune checkpoint inhibitor, or a method for assisting prediction of prognosis of a subject suffering from cancer who has received treatment with an immune checkpoint inhibitor. The method for predicting prognosis of the present invention leads to application of a drug to a cancer patient for which a therapeutic effect of an immune checkpoint inhibitor can be expected, and thus the present invention also contributes to reduction of medical costs and improvement of patient QOL.

### <<Biomarker>>

According to a third aspect of the present invention, there are provided a biomarker used for prediction, determination, or diagnosis of responsiveness to an immune checkpoint inhibitor, the biomarker including the IL-1 signaling pathway molecule of the present invention, and use of the IL-1 signaling pathway molecule of the present invention as a biomarker for prediction, determination, or diagnosis of responsiveness to an immune checkpoint inhibitor. According to the present invention, there is also provided use of the IL-1 signaling pathway molecule of the present invention used as a biomarker in the method for predicting responsiveness of the present invention.

According to the present invention, there are also provided a biomarker used for prediction of prognosis of a subject suffering from cancer who has received treatment with an immune checkpoint inhibitor, the biomarker including the IL-1 signaling pathway molecule of the present invention, and use of the IL-1 signaling pathway molecule of the present invention as a biomarker used for prediction of prognosis of a subject suffering from cancer who has received treatment with an immune checkpoint inhibitor. According to the present invention, there is also provided use of the IL-1 signaling pathway molecule of the present invention used as a biomarker in the method for predicting prognosis of the present invention.

The biomarker of the present invention and use thereof can be implemented according to the description of the method for predicting responsiveness of the present invention and the method for predicting prognosis of the present invention.

In the present invention, the “biomarker” refers to a biologically derived substance whose presence and amount serve as an indicator of responsiveness to an immune checkpoint inhibitor, and can be used as a marker for predicting, identifying, evaluating, determining, and the like therapeutic responsiveness. That is, according to the present invention, the IL-1 signaling pathway molecule of the present invention can be used as an identification marker of therapeutic responsiveness to an immune checkpoint inhibitor.

### <<Diagnostic Kit>>

According to a fourth aspect of the present invention, there are provided a kit used for prediction of responsiveness to an immune checkpoint inhibitor and a kit used for prediction of prognosis of a subject suffering from cancer who has received treatment with an immune checkpoint inhibitor, each kit including a means for quantifying an amount or concentration of an IL-1 signaling pathway molecule in a biological sample. The kit of the present invention can be implemented according to the method for predicting responsiveness to an immune checkpoint inhibitor of the present invention and the method for predicting prognosis of a subject suffering from cancer who has received treatment with an immune checkpoint inhibitor. Examples of the means for quantifying an amount or concentration of an IL-1 signaling pathway molecule in a biological sample include those described as means for measuring the IL-1 signaling pathway molecule of the present invention.

### <<Method for Treating Cancer>>

According to a fifth aspect of the present invention, there is provided a method for treating cancer in a subject predicted to be responsive to treatment with an immune checkpoint inhibitor. This method for treating cancer may include a step of performing the method for predicting responsiveness according to the present invention before starting treatment with an immune checkpoint inhibitor and selecting a subject predicted to be responsive (or expected to be responsive) to treatment with an immune checkpoint inhibitor. This step may include obtaining a test sample from a patient having cancer, measuring an amount or concentration of the IL-1 signaling pathway molecule in the sample, and/or comparing the amount or concentration of the IL-1 signaling pathway molecule in the sample with a cutoff value. When a measurement target is the IL-1 signaling pathway molecule (a), an amount or concentration of the IL-1 signaling pathway molecule (a) in the test sample of the subject before starting treatment with an immune checkpoint inhibitor being higher than a cutoff value indicates that the subject is responsive to the immune checkpoint inhibitor. When a measurement target is the IL-1 signaling pathway molecule (b), an amount or concentration of the IL-1 signaling pathway molecule (b) in the test sample of the subject before starting treatment with an immune checkpoint inhibitor being lower than a cutoff value indicates that the subject is responsive to the immune checkpoint inhibitor

The method for treating cancer may include a step of subjecting a subject predicted to be responsive to treatment with an immune checkpoint inhibitor to treatment with an immune checkpoint inhibitor. The treatment with an immune checkpoint inhibitor is known, and those described in the method for predicting responsiveness of the present invention can be used.

According to the fifth aspect of the present invention, there is also provided a method for treating cancer in a subject undergoing treatment with an immune checkpoint inhibitor. This method for treating cancer may include a step of performing the method for predicting responsiveness according to the present invention after starting treatment with an immune checkpoint inhibitor and selecting a subject predicted to be non-responsive (or expected to be non-responsive) to treatment with an immune checkpoint inhibitor. This step may include obtaining a test sample from a patient having cancer, measuring an amount or concentration of the IL-1 signaling pathway molecule in the sample, and/or comparing the amount or concentration of the IL-1 signaling pathway molecule in the sample with a cutoff value. When a measurement target is the IL-1 signaling pathway molecule (a), an amount or concentration of the IL-1 signaling pathway molecule (a) in the test sample of the subject after starting treatment with an immune checkpoint inhibitor being lower than a cutoff value indicates that the subject is non-responsive to the immune checkpoint inhibitor. When a measurement target is the IL-1 signaling pathway molecule (b), an amount or concentration of the IL-1 signaling pathway molecule (b) in the test sample of the subject after starting treatment with an immune checkpoint inhibitor being higher than a cutoff value indicates that the subject is non-responsive to the immune checkpoint inhibitor.

The method for treating cancer may include a step of subjecting a subject predicted to be non responsive to treatment with an immune checkpoint inhibitor to treatment other than the treatment with an immune checkpoint inhibitor. Treatment of cancer other than the treatment with an immune checkpoint inhibitor is known, and examples thereof include other therapies other than the immune checkpoint inhibitor, such as chemotherapy, immunotherapy, radiation therapy, and surgery, and also include palliative therapy such as palliative care.

The method for treating cancer of the present invention can be carried out according to the description of the method for predicting responsiveness of the present invention. In particular, the determination on whether or not a subject is responsive to an immune checkpoint inhibitor and the determination on whether or not a subject is non-responsive to an immune checkpoint inhibitor can be carried out according to the contents described in the method for predicting responsiveness of the present invention. In the method for treating cancer of the present invention, a plurality of kinds of IL-1 signaling pathway molecules of the present invention may be combined and used as an indicator, and such an embodiment can be carried out according to the content described in the method for predicting responsiveness of the present invention.

### EXAMPLES

The present invention will be described more specifically based on the following examples; however, the present invention is not limited to these examples.

### Statistical Analysis

All animal experimental data were expressed as mean±SD of six independent tests. Statistical analysis was performed using the student t-test and dispersion assay (ANOVA)+Tukey-Kramer Post-hoc test as appropriate. For the measurement data of patient serum samples, all the measurement data were plotted. Comparison between responders and non-responders was performed using Welch t-test.

### Approval of Study

All the animal experiment procedures were performed with the approval of the Institutional Animal Care and Use Committee of Graduate School of Medicine, The University of Tokyo. The experiment using the human specimen was performed in accordance with a research protocol approved by the Ethics Committee of Graduate School of Medicine, The University of Tokyo.

### Example 1: Temporal Changes in Serum Proteins in LLC Tumor-Bearing Mice

In order to evaluate the responsiveness to the immune checkpoint inhibitor, in vivo changes due to proliferation of Lewis lung cancer (LLC) known as a cancer having low responsiveness to the anti-PD-1 antibody were examined. Specifically, LLC tumor tumor-bearing mice were prepared, serum was collected, and biomarkers that change with proliferation of LLC were identified by quantitative proteomics.

### (1) Cell Culture

LLC cells were cultured in DMEM (nacalai tesque) supplemented with 10% fetal bovine serum (FBS, Biowest) and 1% penicillin streptomycin (PCSM, Life Technologies).

### (2) Preparation of Tumor-Bearing Mice

C57BL/6 mice used for the experiment were purchased from Japan SLC, Inc., acclimated for a minimum of 7 days, and then used for the experiment at 7 weeks of age. The LLC cells were subcutaneously injected as 2×10⁵ cells each into back of C57BL/6 mice to prepare tumor-bearing mice (test group, n=6). Whole blood was collected 7, 14, and 21 days after subcutaneous injection, and serum was collected by centrifugation. On the other hand, the mice in the control group were treated in the same manner as in the test group except that the cells were not transplanted.

### (3) Identification of Serum Proteins by Proteomics Analysis

Pretreatment with Albumin/IgG removal kit (CALBIOCHEM) was performed in order to remove albumin and IgG contained in a large amount in the serum and to facilitate the measurement of proteins in a trace amount. Serum samples were then reductively alkylated using dithiothreitol (DTT, nacalai tesque) and iodoacetamide (FUJIFILM Wako Pure Chemical Corporation), followed by precipitation recovery of protein fractions using 10-fold amount of acetone (nacalai tesque). The precipitate fraction was redissolved in a 100 mM triethylammonium hydrogen carbonate solution (FUJIFILM Wako Pure Chemical Corporation), digested with trypsin/Lys-C Mix (Promega), and the digested sample was collected on an SDB column (styrenedivinylbenzene column; GL Sciences Inc.) and a GC column (graphite carbon column; GL Sciences Inc.) to selectively extract peptides. The extract was dried to solid with a speed bag to prepare a sample for proteomics. In the proteomics analysis, LC/MS analysis was performed using a high-resolution mass spectrometer (Q Exactive™, Thermo Scientific), and protein identification and label-free quantification of the obtained mass spectrometry data were performed using Proteome Discoverer software (Thermo Scientific).

### (4) Measurement of Serum Protein Levels

Estimation of concentration levels of candidate proteins contained in serum samples was performed by a label-free quantification method using Proteome Discoverer software (Thermo Scientific).

### (5) Results

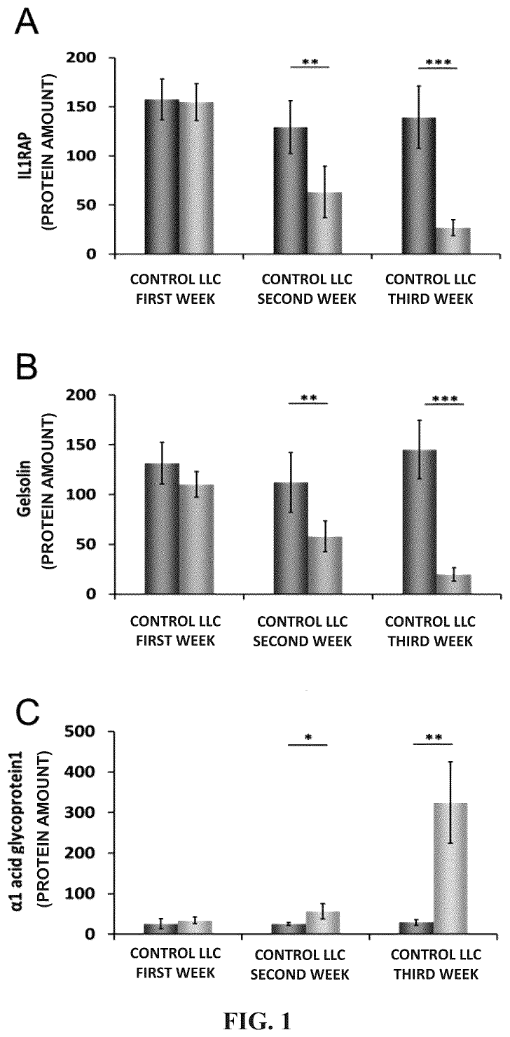

The results were as shown in FIG. 1. IL-1RAP, Gelsolin, and α1 acid glycoprotein 1 were identified as biomarker candidate substances that change with the proliferation of LLC. The serum levels of IL-1RAP and Gelsolin decreased over time in the LLC tumor-bearing group, with a significant difference observed at the second week and the third week (FIGS. 1A and 1B). Gelsolin decreased over time, and a significant difference was observed between the second week and the third week (FIG. 1B). α1 acid glycoprotein 1 increased over time, and a significant difference was observed between the second week and the third week (FIG. 1C).

### Example 2: Concentration Variations of Serum Proteins in LLC. MC38, or B16F10 Tumor-Bearing

In order to determine whether IL-1RAP, Gelsolin, and α1 acid glycoprotein 1 are involved or not in responsiveness to an immune checkpoint inhibitor, tumor-bearing mice were prepared using MC38 (mouse colorectal cancer cell line, Russell W. Jenkin et al, Cancer Discov. 2018; 8(2):196-215.) known as a cancer having a higher responsiveness to an anti-PD-1 antibody compared to LLC and B16F10 (mouse malignant melanoma cell line, Elizabeth Ahern et al, Oncoimmunology. 2018; 7(6): e1431088.) known as a cancer having a low therapeutic responsiveness similar to LLC, and the concentrations of IL-1RAP, Gelsolin, and α1 acid glycoprotein 1 in the serum were examined.

### (1) Cell Culture

LLC and MC38 cells were cultured in DMEM (nacalai tesque) supplemented with 10% fetal bovine serum (FBS, Biowest) and 1% penicillin streptomycin (PCSM, Life Technologies). B16F10 cells were cultured in RPM (nacalai tesque) supplemented with 10% FBS, 2 mM L-glutamine (nacalai tesque), and 1% PCSM.

### (2) Preparation of Tumor-Bearing Mice

tumor-bearing mice (n=6 for each) were prepared in the same manner as in (2) of Example 1 except that LLC, MC38, and B16F10 cells were used, and whole blood was collected 18 days after subcutaneous injection of the cells.

### (3) Measurement of Serum Protein Concentration

The mouse IL-1RAP concentration was measured using ELISA Kit for Interleukin 1 Receptor Accessory Protein (IL-TRAP) (Cloud-Clone), the mouse Gelsolin concentration was measured using ELISA Kit for Gelsolin (GSN) (Cloud-Clone), and the mouse α1 acid glycoprotein 1 concentration was measured using Alpha-1 Acid Glycoprotein 1 (Mouse) ELISA Kit (Biovision), according to the protocol, respectively.

### (4) Results

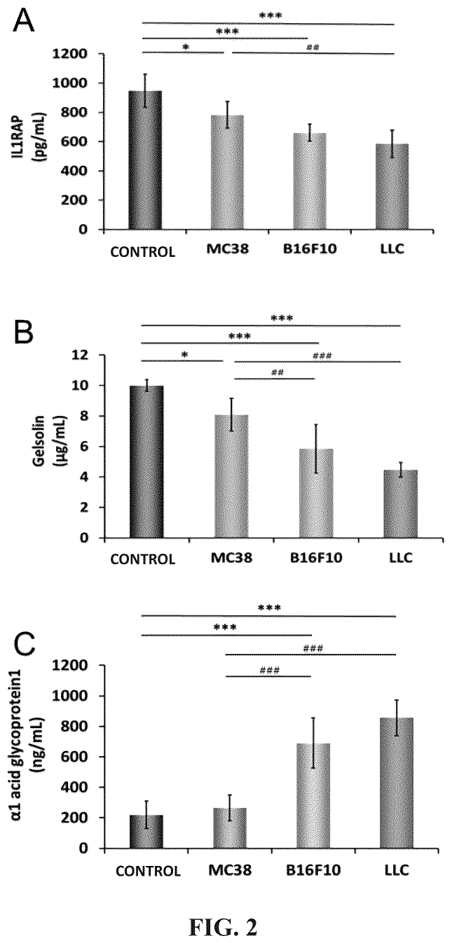

The results were as shown in FIG. 2. The concentrations of IL-1RAP, Gelsolin, and α1 acid glycoprotein 1 varied greatly in the LLC or B16F10 tumor-bearing mice with low therapeutic responsiveness as compared to the MC38 tumor-bearing mice with high responsiveness to the anti-PD-1 antibody. Specifically, IL-TRAP decreased more in B16F10 and LLC compared to MC38 (FIG. 2A), Gelsolin decreased more in B16F10 and LLC compared to MC38 (FIG. 2B), and α1 acid glycoprotein 1 increased more in B16F10 and LLC compared to MC38 (FIG. 2C). From these results, it was shown that IL-TRAP, Gelsolin, and α1 acid glycoprotein 1 proteins may be involved in responsiveness to an immune checkpoint inhibitor.

### Example 3: IL-TRAP Correlates with Responsiveness to Immune Checkpoint Inhibitor (1)

A clinical study was conducted to determine the correlation of the biomarker candidate substances (IL-1RAP, Gelsolin, and α1 acid glycoprotein 1) identified in Examples 1 and 2 with responsiveness to an immune checkpoint inhibitor.

### (1) Clinical Observation Test

As a standard of care for the progression or recurrence of lung cancer or renal cancer, 50 patients who received an immune checkpoint inhibitor were subjects for observation test. The informed consent in writing was obtained from each patient, and blood samples were collected periodically from immediately before starting treatment to the end of treatment, and classified into a good with good treatment response (response group) and a group with poor treatment response (non-response group). Information on the therapeutic effect on the patient's drug was obtained from the patient's chart.

### (2) Measurement of Serum Protein Concentration

The human IL1RAP concentration was measured using Human IL-1 R3/IL-1R Acp ELISA (Ray Biotech), the human Gelsolin concentration was measured using Human Gelsolin ELISA Kit (abcam), and the human α1 acid glycoprotein 1 concentration was measured using Human Alpha-1-acid glycoprotein 1 ELISA kit (CUSABIO), according to the product protocol, respectively.

### (3) ROC Analysis

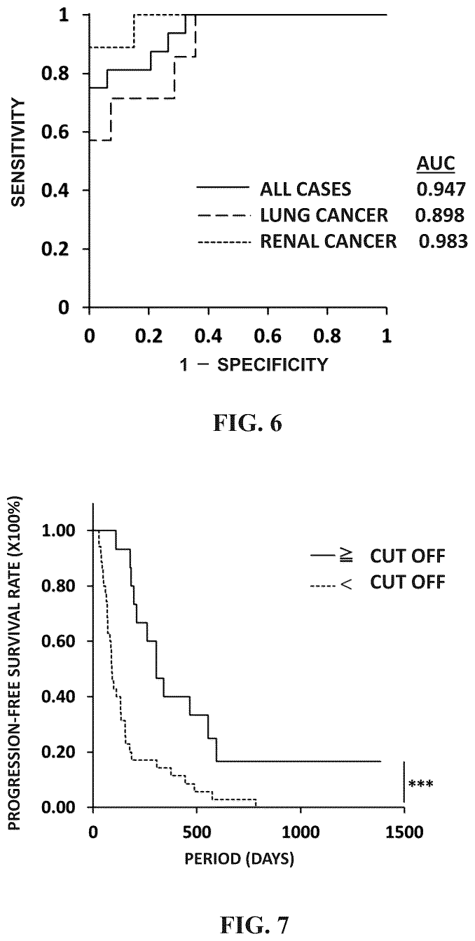

The discrimination between the response group and the non-response group for the IL-1RAP protein was analyzed by an ROC curve (Table 1 and FIG. 6). Excel statistics as statistical analysis software was used for these analyses.

### (4) Analysis of Progression-Free Survival Rate

Kaplan-Meier curves were plotted using Excel statistics as statistical analysis software.

TABLE 1
Evaluation of therapeutic responsiveness prediction accuracy
for immune checkpoint inhibitor-administered patients using
IL-1RAP concentration before starting treatment

| | All cases | Lung cancer | Renal cancer |
|---|---|---|---|
| ROC | FIG. 7 | FIG. 7 | FIG. 7 |
| AUC | 0.947 | 0.898 | 0.983 |
| Cutoff value | 154.35 | 154.35 | 168.53 |
| Sensitivity (%) | 81.25 | 66.67 | 88.89 |
| Specificity (%) | 97.06 | 92.86 | 100.00 |
| Positive predictive value (%) | 92.86 | 80.00 | 100.00 |
| Negative predictive value (%) | 91.67 | 86.67 | 95.24 |

### (5) Results

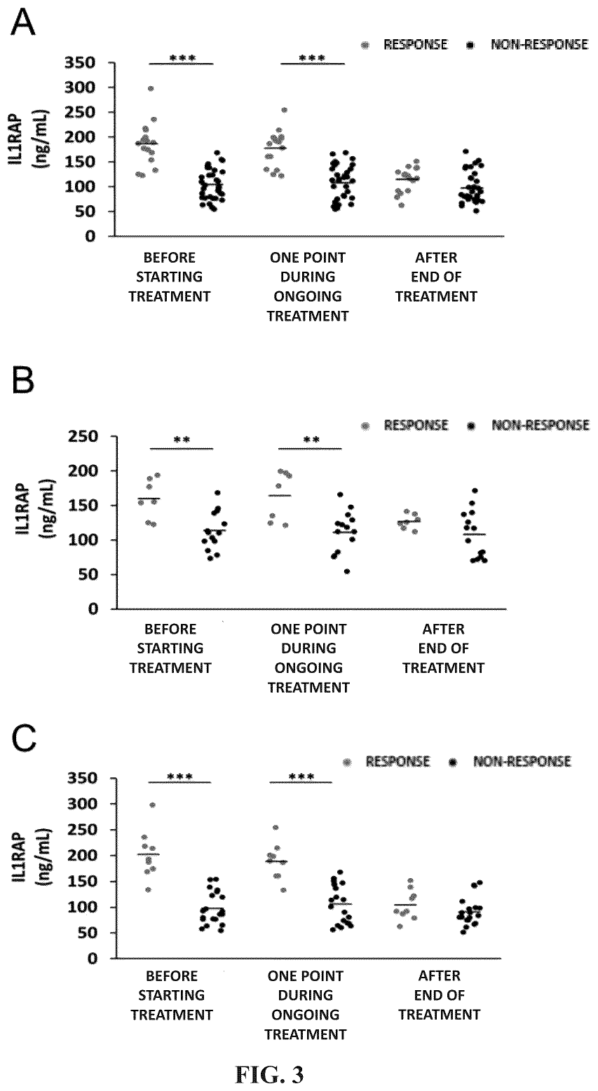

The results were as shown in Table 1 and FIGS. 3 to 7.

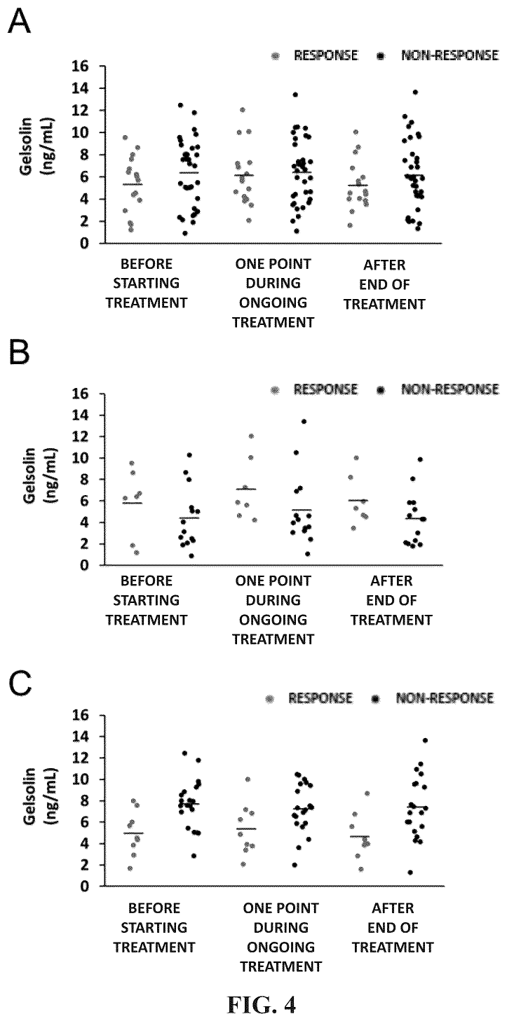

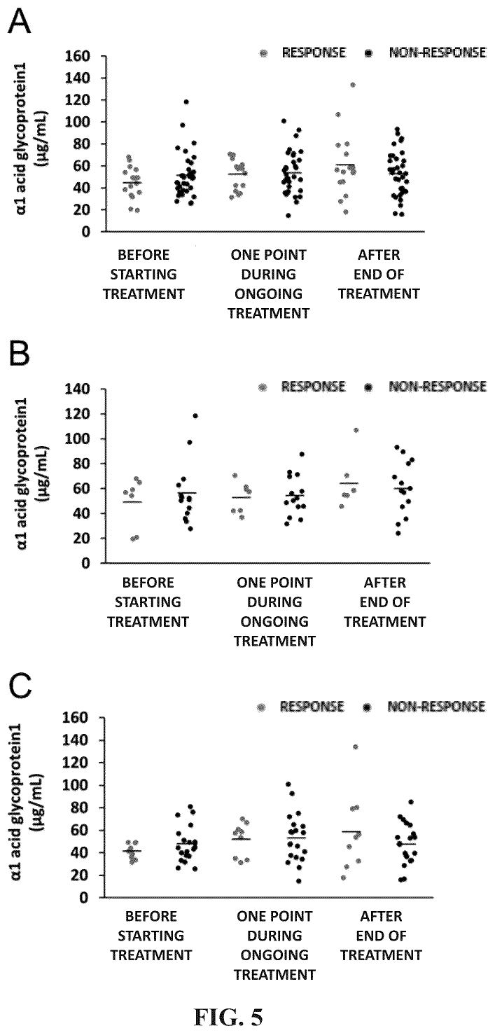

From FIG. 3, it was shown that the IL-1RAP concentration was significantly higher in the response group than in the non-response group from before starting treatment. As a result of ROC analysis using the IL-1RAP concentration before starting treatment, the obtained AUC values were 0.947 in all cases, 0.898 in lung cancer cases, and 0.983 in renal cancer cases, and it was shown that the IL-1RAP concentration before starting treatment can accurately separate the response group and the non-response group (Table 1 and FIG. 6). From these results, it was shown that responsiveness to an immune checkpoint inhibitor can be predicted using the IL-1RAP concentration in blood (serum) before starting treatment as an indicator. From FIGS. 4 and 5, the concentrations of Gelsolin and α1 acid glycoprotein 1 were not correlated with responsiveness to an immune checkpoint inhibitor.

From FIG. 3, it was also shown that the IL-1 RAP concentration was significantly higher in the response group than in the non-response group even after starting treatment (one point during administration continuation). From this result, it was shown that responsiveness to an immune checkpoint inhibitor can be predicted using the IL-TRAP concentration in blood (serum) after starting treatment as an indicator.

From FIG. 3, it also became clear that the IL-1RAP concentration decreased to the same level as that in the non-response group at a stage where exacerbation of tumor was observed and therapeutic resistance to an immune checkpoint inhibitor was shown (timing of invalidation) in the response group. From this result, it was shown that responsiveness (including therapeutic non-responsiveness, that is, therapeutic resistance) to an immune checkpoint inhibitor can be predicted using the IL-1RAP concentration in blood (serum) after starting treatment as an indicator. It is also possible to periodically measure the IL-1RAP concentration in the serum even after the start of administration of the immune checkpoint inhibitor, and assist the timing of switching to the post-treatment when the IL-TRAP concentration decreases.

From FIG. 7, it was shown that in the group in which the IL-1RAP concentration before starting treatment was higher than the cutoff value, the progression-free survival rate for all cases was significantly higher as compared with the group in which the IL-TRAP concentration before starting treatment was lower than the cutoff value. From these results, it was shown that the prognosis of cancer can be predicted using the IL-1 RAP concentration in blood (serum) before starting treatment as an indicator.

### Example 4: IL-1RAP Correlates with Responsiveness to Immune Checkpoint Inhibitor (2)

### (1) Clinical Observation Test

The clinical observation test was performed in the same manner as in (1) of Example 3.

### (2) Measurement of Serum Protein Concentration

The human IL-1RAP concentration was measured using Human IL-1 R3/IL-1R Acp ELISA (Catalog No. ELH-IL1R3-1, Ray Biotech) according to the protocol.

### (3) ROC Analysis

The discrimination between the response group and the non-response group for the IL-1RAP protein was analyzed by an ROC curve. These analyses were performed by the inventors using Python according to a conventional method. The cutoff value was determined by searching for a point on the ROC curve that is the shortest distance from a point (upper left point on the graph) at which the value on the horizontal axis is designated as “0” and the value on the vertical axis is designated as “1”.

### (4) Analysis of Progression-Free Survival Rate

The progression-free survival rate was plotted on the Kaplan-Meier curve with 95% confidence intervals using Python's lifelines library (P<0.005 between groups with a significant difference, log-rank test).

TABLE 2
Therapeutic responsiveness prediction accuracy for immune checkpoint
inhibitor-administered patients in case of using cutoff value of
ROC curve of IL-1RAP concentration before starting treatment

| | All | Lung cancer | Renal cancer |
|---|---|---|---|
| | cases | cases | cases |
| Sensitivity | 0.812500 | 0.714286 | 0.888889 |
| Specificity | 0.941176 | 0.928571 | 0.950000 |
| Positive predictive value | 0.866667 | 0.833333 | 0.888889 |
| Negative predictive value | 0.914286 | 0.866667 | 0.950000 |

### (5) Results

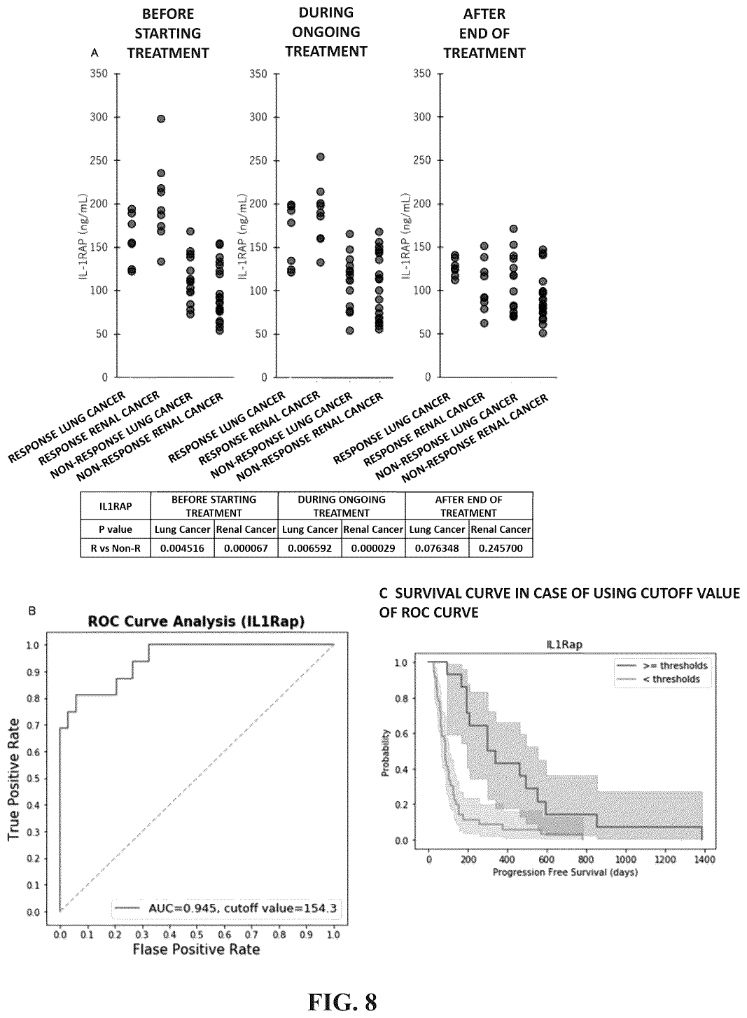

The results were as shown in Table 2 and FIG. 8.

From FIG. 8A, it was shown that the IL-1RAP concentration was significantly higher in the response group than in the non-response group from before starting treatment. From FIG. 8B, as a result of ROC analysis using the IL-TRAP concentration before starting treatment, it was shown that the CL-1 RAP concentration before starting treatment can accurately separate the response group and the non-response group (Table 2). From these results, it was shown that therapeutic responsiveness to an immune checkpoint inhibitor can be predicted using the IL-1RAP concentration in blood (serum) before starting treatment as an indicator.

From FIG. 8A, it was also shown that the IL-1 RAP concentration was significantly higher in the response group than in the non-response group even after starting treatment (one point during ongoing treatment). From this result, it was shown that responsiveness to an immune checkpoint inhibitor can be predicted using the IL-1 RAP concentration in blood (serum) after starting treatment as an indicator.

From FIG. 8A, it also became clear that the IL-1 RAP concentration decreased to the same level as that in the non-response group at a stage where exacerbation of tumor was observed and therapeutic resistance to an immune checkpoint inhibitor was shown (timing of invalidation) in the response group. From this result, it was shown that responsiveness (including non-responsiveness, that is, therapeutic resistance and invalidation of an immune checkpoint inhibitor) to an immune checkpoint inhibitor can be predicted using the IL-TRAP concentration in blood (serum) after starting treatment as an indicator.

From FIG. 8C, it was shown that in the group in which the IL-1RAP concentration before starting treatment was higher than the cutoff value, the progression-free survival rate for all cases was significantly higher as compared with the group in which the IL-1RAP concentration before starting treatment was lower than the cutoff value. From these results, it was shown that the prognosis of cancer can be predicted using the IL-TRAP concentration in blood (serum) before starting treatment as an indicator.

### Example 5: IL-1R2 Correlates with Responsiveness to Immune Checkpoint Inhibitor

A clinical study was conducted to determine the correlation of IL-TRAP and functionally related IL-1R2 with responsiveness to an immune checkpoint inhibitor.

### (1) Clinical Observation Test

The clinical observation test was performed in the same manner as in (1) of Example 3.

### (2) Measurement of Serum Protein Concentration

The human IL-1R2 concentration was measured using Human IL-1 RII Quantikine ELISA Kit (Catalog No. DR1B00, R&D Systems) according to the protocol.

### (3) ROC Analysis

ROC analysis was performed in the same manner as in (3) of Example 4.

### (4) Analysis of Progression-Free Survival Rate

The progression-free survival rate was performed in the same manner as in (4) of Example 4.

TABLE 3
Therapeutic responsiveness prediction accuracy for immune checkpoint
inhibitor-administered patients in case of using cutoff value
of ROC curve of IL-1R2 concentration before starting treatment

| | All cases | Lung cancer | Renal cancer |
|---|---|---|---|
| Sensitivity | 0.937500 | 1.000000 | 0.888889 |
| Specificity | 0.970588 | 1.000000 | 0.950000 |
| Positive predictive value | 0.937500 | 1.000000 | 0.888889 |
| Negative predictive value | 0.970588 | 1.000000 | 0.950000 |

TABLE 4
Therapeutic responsiveness prediction accuracy for immune
checkpoint inhibitor-administered patients in case of
using cutoff value of ROC curve of IL-1RAP concentration
and IL-1R2 concentration before starting treatment

| | All cases | Lung cancer | Renal cancer |
|---|---|---|---|
| Sensitivity | 1.000000 | 1.000000 | 1.000000 |
| Specificity | 0.941177 | 0.928571 | 0.950000 |
| Positive predictive value | 0.888889 | 0.875000 | 0.900000 |
| Negative predictive value | 1.000000 | 1.000000 | 1.000000 |

### (5) Results

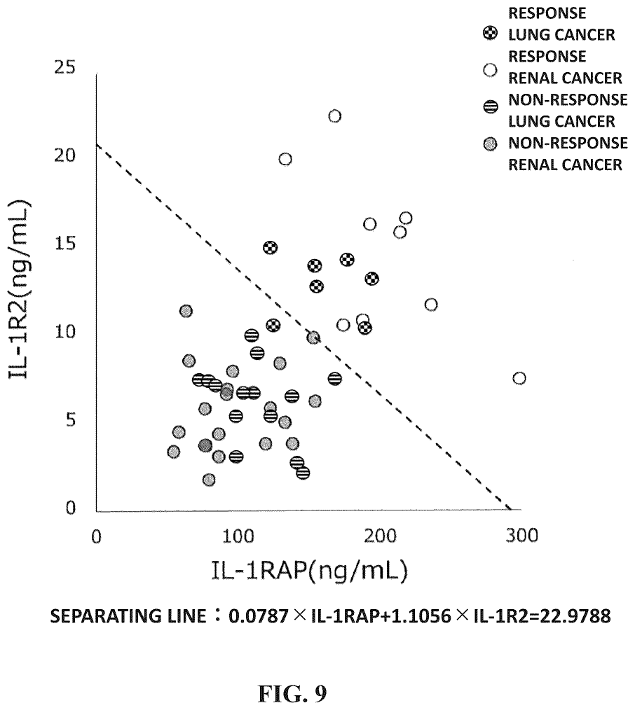

The results were as shown in Tables 3 and 4 and FIGS. 9, 10, and 11.

From FIG. 9, a certain correlation was observed between the concentration of IL-1 R2 and the concentration of IL-1RAP in the serum.

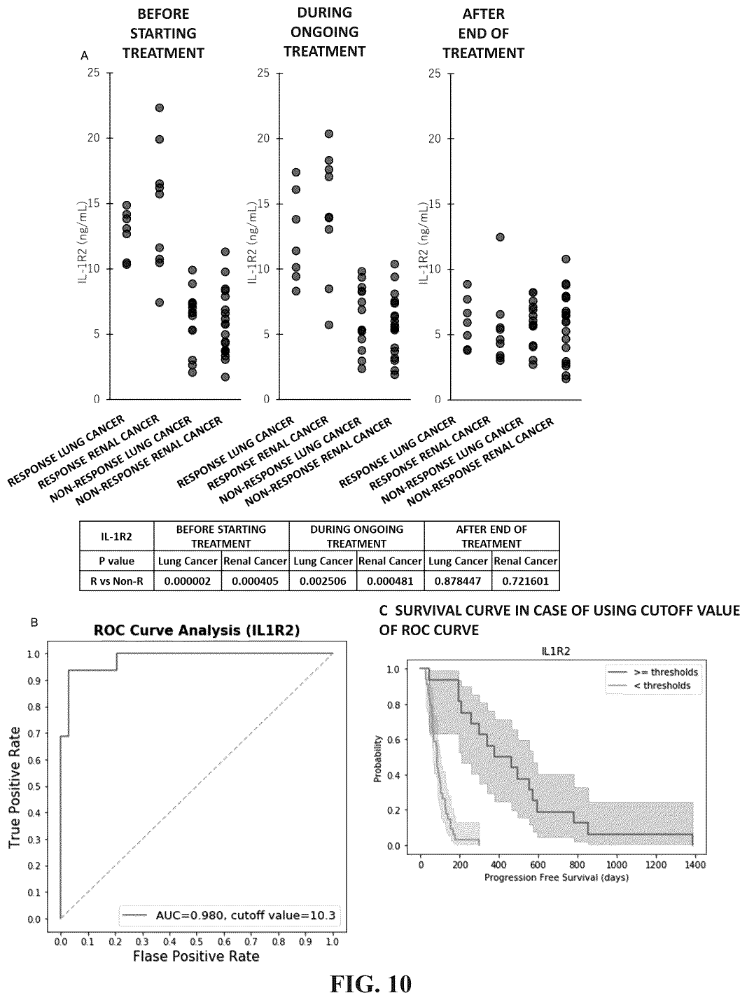

From FIG. 10A, it was shown that the IL-1R2 concentration was significantly higher in the response group than in the non-response group from before starting treatment. From FIG. 10B, as a result of ROC analysis using the IL-1R2 concentration before starting treatment, it was shown that the IL-1R2 concentration before starting treatment can accurately separate the response group and the non-response group (Table 3). From these results, it was shown that therapeutic responsiveness to an immune checkpoint inhibitor can be predicted using the IL-1R2 concentration in blood (serum) before starting treatment as an indicator.

From FIG. 10A, it was also shown that the IL-1R2 concentration was significantly higher in the response group than in the non-response group even after starting treatment (one point during ongoing treatment). From this result, it was shown that responsiveness to an immune checkpoint inhibitor can be predicted using the IL-1R2 concentration in blood (serum) after starting treatment as an indicator.

From FIG. 10A, it also became clear that the IL-1R2 concentration decreased to the same level as that in the non-response group at a stage where exacerbation of tumor was observed and therapeutic resistance to an immune checkpoint inhibitor was shown (timing of invalidation) in the response group. From this result, it was shown that responsiveness (including non-responsiveness, that is, therapeutic resistance and invalidation of an immune checkpoint inhibitor) to an immune checkpoint inhibitor can be predicted using the IL-1R2 concentration in blood (serum) after starting treatment as an indicator.

From FIG. 10C, it was shown that in the group in which the IL-1R2 concentration before starting treatment was higher than the cutoff value, the progression-free survival rate for all cases was significantly higher as compared with the group in which the IL-1R2 concentration before starting treatment was lower than the cutoff value. From these results, it was shown that the prognosis of cancer can be predicted using the IL-1R2 concentration in blood (serum) before starting treatment as an indicator.

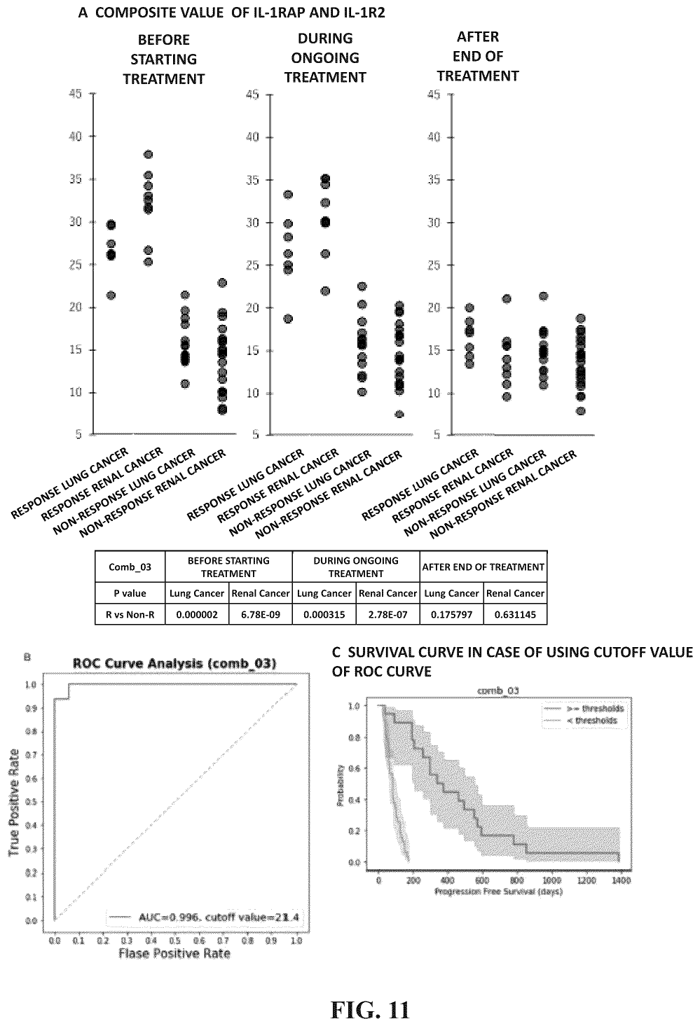

From FIG. 11A, it was shown that the indicator obtained by linearly combining the concentrations of IL-1RAP and IL-1R2 in the serum (0.0787×IL-1RAP+1.1056*IL-1R2) was significantly higher in the response group than in the non-response group from before starting treatment. From FIG. 11B, as a result of ROC analysis using the composite value of IL-1RAP and IL-1 R2 before starting treatment, it was shown that the composite value of IL-1RAP and IL-1R2 before starting treatment can almost completely separate the response group and the non-response group (Table 4). From these results, it was shown that the composite value of IL-1 RAP and IL-1R2 in blood (serum) before starting treatment can predict responsiveness to an immune checkpoint inhibitor.

From FIG. 11A, it was also shown that the composite value of IL-1RAP and IL-1R2 was significantly higher in the response group than in the non-response group even after starting treatment (one point during ongoing treatment). From this result, it was shown that the composite value of IL-1RAP and IL-1R2 in blood (serum) after starting treatment can predict responsiveness to an immune checkpoint inhibitor.

From FIG. 11A, it also became clear that the composite value of IL-TRAP and IL-1R2 decreased to the same level as that in the non-response group at a stage where exacerbation of tumor was observed and therapeutic resistance to an immune checkpoint inhibitor was shown (timing of invalidation) in the response group. From this result, it was shown that the composite value of IL-1RAP and IL-1R2 in blood (serum) after starting treatment can predict responsiveness (including non-responsiveness, that is, therapeutic resistance and invalidation of an immune checkpoint inhibitor) to an immune checkpoint inhibitor.

From FIG. 11C, it was shown that in the group in which the composite value of IL-1RAP and IL-1R2 before starting treatment was higher than the cutoff value, the progression-free survival rate for all cases was significantly higher as compared with the group in which the composite value before starting treatment was lower than the cutoff value. From these results, it was shown that the prognosis of cancer can be predicted using the composite value of IL-TRAP and IL-1R2 in blood (serum) before starting treatment as an indicator.

### Example 6: IL-1β Correlates with Responsiveness to Immune Checkpoint Inhibitor

A clinical study was conducted to determine the correlation of IL-1β trapped in IL-1R2 and IL-1RAP with responsiveness to an immune checkpoint inhibitor.

### (1) Clinical Observation Test

The clinical observation test was performed in the same manner as in (1) of Example 3.

### (2) Measurement of Serum Protein Concentration

The human IL-1β concentration was measured using Human IL-1 beta/IL-1F2 Quantikine ELISA Kit (Catalog No. DLB50, R&D Systems) according to the protocol.

### (3) ROC Analysis

ROC analysis was performed in the same manner as in (3) of Example 4.

### (4) Analysis of Progression-Free Survival Rate

The progression-free survival rate was performed in the same manner as in (4) of Example 4.

TABLE 5
Therapeutic responsiveness prediction accuracy for immune checkpoint
inhibitor-administered patients in case of using cutoff value of
ROC curve of IL-1β concentration before starting treatment

| | All cases | Lung cancer | Renal cancer |
|---|---|---|---|
| Sensitivity | 0.937500 | 1.000000 | 0.888889 |
| Specificity | 0.911765 | 0.928571 | 0.900000 |
| Positive predictive value | 0.833333 | 0.875000 | 0.800000 |
| Negative predictive value | 0.968750 | 1.000000 | 0.947368 |

TABLE 6
Therapeutic responsiveness prediction accuracy for immune checkpoint
inhibitor-administered patients in case of using cutoff value
of ROC curve of composite value of IL-1RAP concentration and
IL-1β concentration before starting treatment

| | All cases | Lung cancer | Renal cancer |
|---|---|---|---|
| Sensitivity | 1.000000 | 1.000000 | 1.000000 |
| Specificity | 0.941177 | 0.928571 | 0.950000 |
| Positive predictive value | 0.888889 | 0.875000 | 0.900000 |
| Negative predictive value | 1.000000 | 1.000000 | 1.000000 |

TABLE 7
Therapeutic responsiveness prediction accuracy for immune checkpoint
inhibitor-administered patients in case of using cutoff value
of ROC curve of composite value of IL-1β concentration
and IL-1R2 concentration before starting treatment

| | All cases | Lung cancer | Renal cancer |
|---|---|---|---|
| Sensitivity | 1.000000 | 1.000000 | 1.000000 |
| Specificity | 0.970588 | 0.928571 | 1.000000 |
| Positive predictive value | 0.941177 | 0.875000 | 1.000000 |
| Negative predictive value | 1.000000 | 1.000000 | 1.000000 |

TABLE 8
Therapeutic responsiveness prediction accuracy for
immune checkpoint inhibitor-administered patients
in case of using cutoff value of ROC curve of composite
value between IL-1RAP and IL-1R2 concentrations and
IL-1β concentration before starting treatment

| | All cases | Lung cancer | Renal cancer |
|---|---|---|---|
| Sensitivity | 1.000000 | 1.000000 | 1.000000 |
| Specificity | 1.000000 | 1.000000 | 1.000000 |
| Positive predictive value | 1.000000 | 1.000000 | 1.000000 |
| Negative predictive value | 1.000000 | 1.000000 | 1.000000 |

### (4) Results

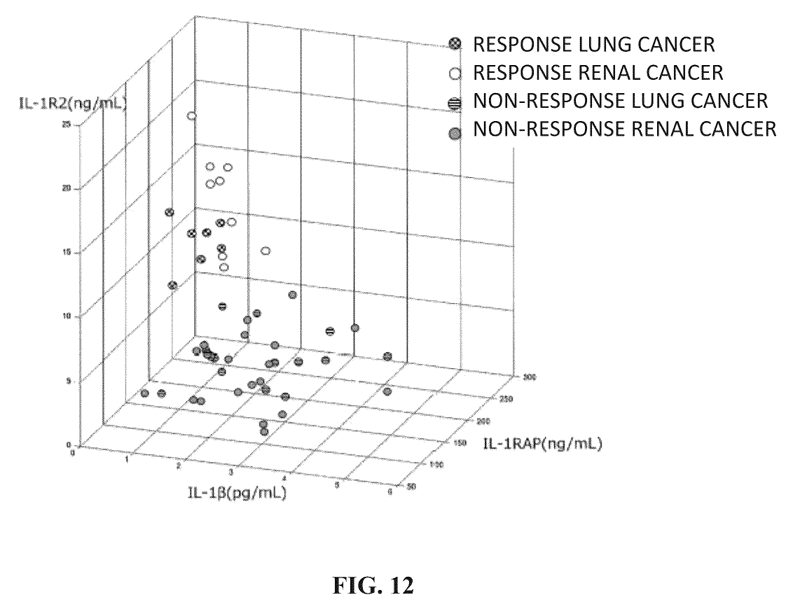

The results were as shown in Tables 5 to 8 and FIGS. 12 to 17.

From FIG. 12, a certain correlation was observed between the IL-1β concentration in the serum and changes in the concentrations of IL-1RAP and IL-1R2.

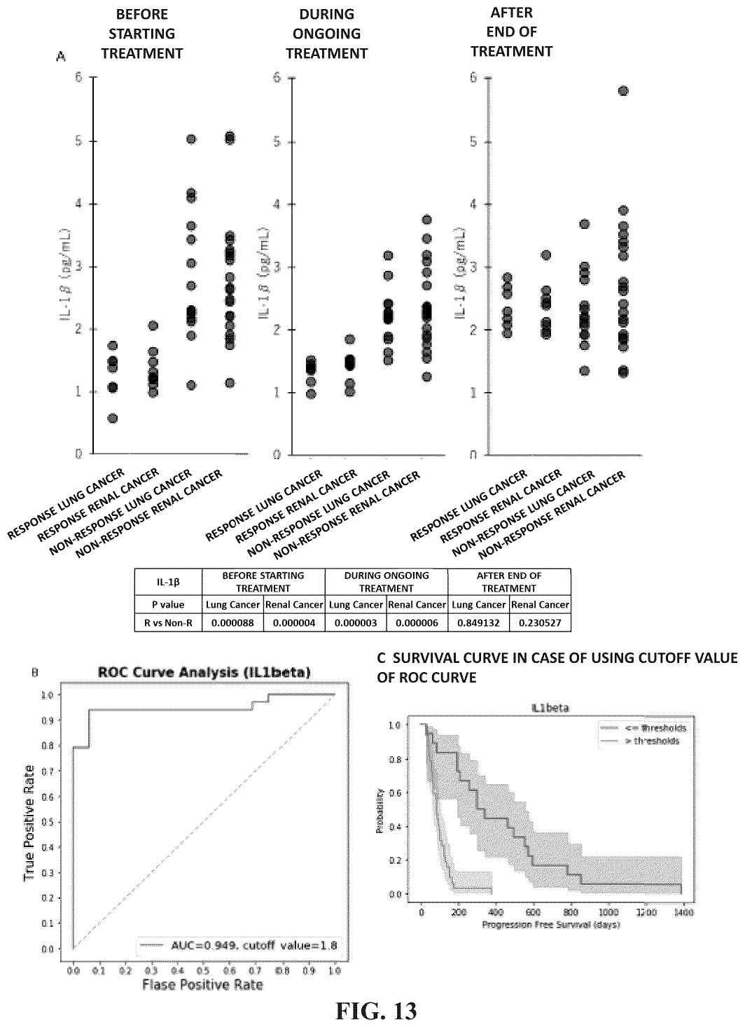

From FIG. 13A, it was shown that the IL-1β concentration was significantly lower in the response group than in the non-response group from before starting treatment. From FIG. 13B, as a result of ROC analysis using the IL-1β concentration before starting treatment, it was shown that the IL-1β concentration before starting treatment can accurately separate the response group and the non-response group (Table 5). From these results, it was shown that responsiveness to an immune checkpoint inhibitor can be predicted using the IL-1β concentration in blood (serum) before starting treatment as an indicator.

From FIG. 13A, it was also shown that the IL-1β concentration was significantly lower in the response group than in the non-response group even after starting treatment (one point during ongoing treatment). From this result, it was shown that responsiveness to an immune checkpoint inhibitor can be predicted using the IL-1β concentration in blood (serum) after starting treatment as an indicator.

From FIG. 13A, it also became clear that the IL-1β concentration increased to the same level as that in the non-response group at a stage where exacerbation of tumor was observed and therapeutic resistance to an immune checkpoint inhibitor was shown (timing of invalidation) in the response group. From this result, it was shown that responsiveness (including non-responsiveness, that is, therapeutic resistance and invalidation of an immune checkpoint inhibitor) to an immune checkpoint inhibitor can be predicted using the IL-1β concentration in blood (serum) after starting treatment as an indicator.

From FIG. 13C, it was shown that in the group in which the IL-1β concentration before starting treatment was lower than the cutoff value, the progression-free survival rate for all cases was significantly higher as compared with the group in which the IL-1β concentration before starting treatment was higher than the cutoff value. From these results, it was shown that the prognosis of cancer can be predicted using the IL-1β concentration in blood (serum) before starting treatment as an indicator.

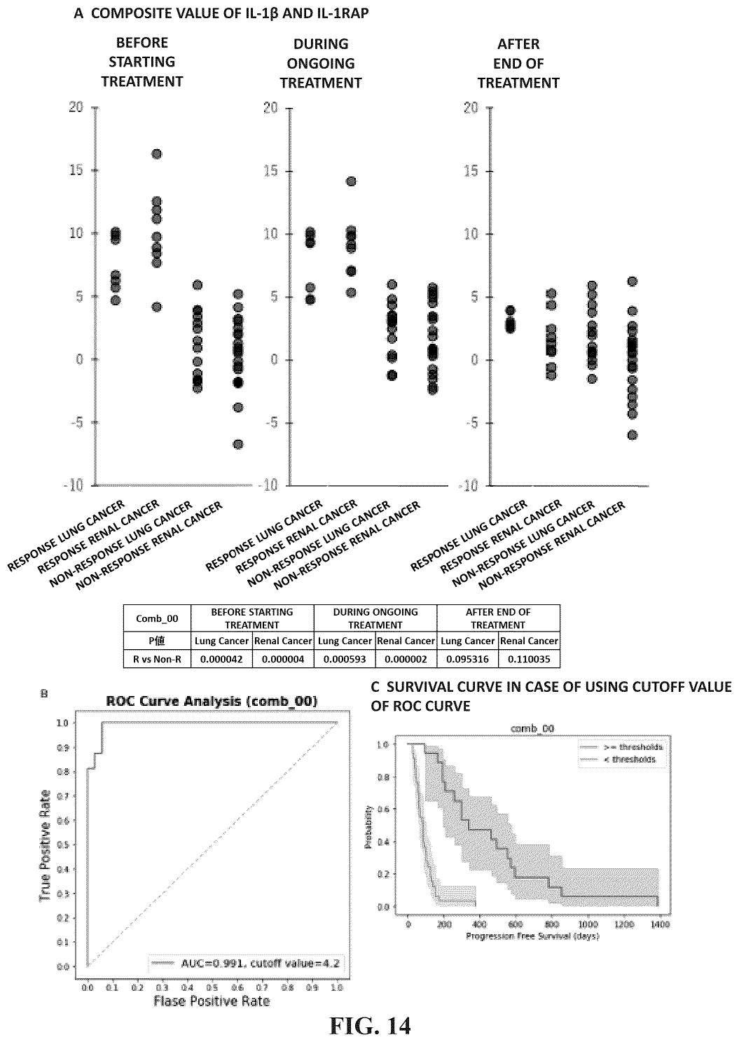

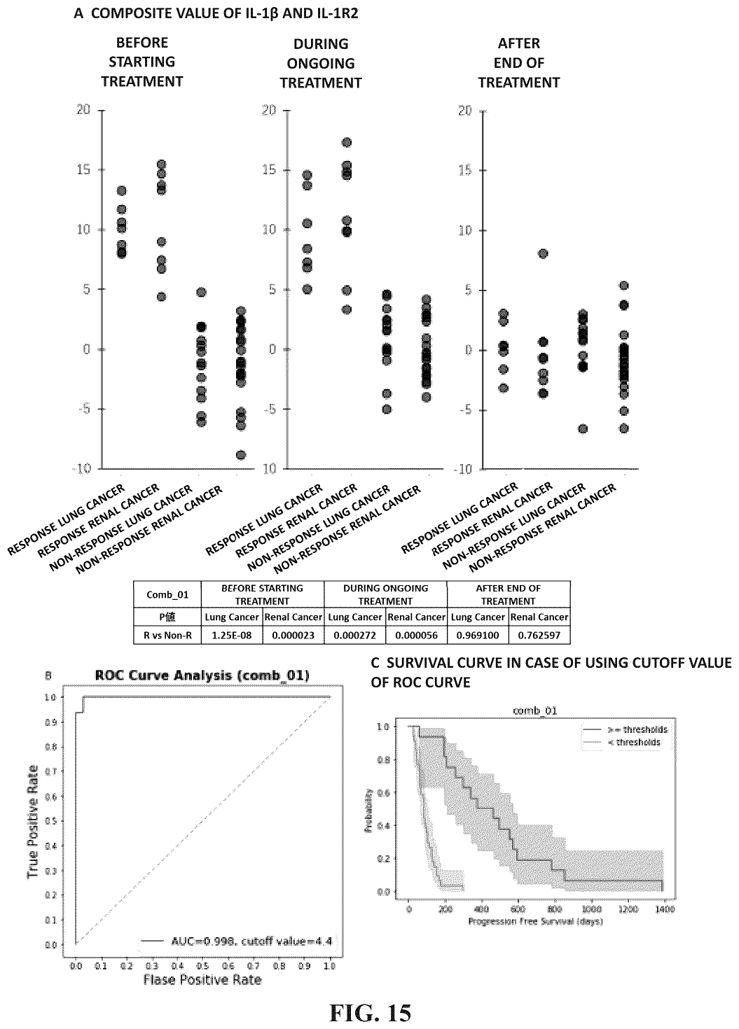

From FIGS. 14A and 15A, it was shown that the indicator obtained by linearly combining the concentrations of IL-1β and IL-1 RAP in the serum (−2.1178 IL-1β+0.062×IL-1 RAP) and the indicator obtained by linearly combining the concentrations of IL-1β and IL-1R2 in the serum (−2.5337×IL-1β+1.04×IL-1R2) were significantly higher in the response group than in the non-response group from before starting treatment. From FIGS. 14B and 15B, as a result of ROC analysis using the composite value of IL-1β and IL-1RAP and the composite value of IL-1β and IL-1R2, the composite value of IL-1β and IL-1RAP and the composite value of IL-1β and IL-1R2 before starting treatment can accurately separate the response group and the non-response group (Tables 6 and 7). From these results, it was shown that the composite value of IL-1β and IL-1 RAP and the composite value of IL-1β and IL-1R2 in blood (serum) before starting treatment can predict responsiveness to an immune checkpoint inhibitor.

From FIGS. 14A and 15A, it was also shown that the composite value of IL-1β and IL-1RAP and the composite value of IL-1β and IL-1 R2 were significantly higher in the response group than in the non-response group even after starting treatment (one point during ongoing treatment). From this result, it was shown that the composite value of IL-1β and IL-1RAP and the composite value of IL-1β and IL-1R2 in blood (serum) after starting treatment can predict responsiveness to an immune checkpoint inhibitor.

From FIGS. 14A and 15A, it also became clear that the composite value of IL-1β and IL-1RAP and the composite value of IL-1β and IL-1R2 decreased to the same level as that in the non-response group at a stage where exacerbation of tumor was observed and therapeutic resistance to an immune checkpoint inhibitor was shown (timing of invalidation) in the response group. From this result, it was shown that the composite value of IL-1β and IL-1RAP and the composite value of IL-1β and IL-1 R2 in blood (serum) after starting treatment can predict responsiveness (including non-responsiveness, that is, therapeutic resistance and invalidation of an immune checkpoint inhibitor) to an immune checkpoint inhibitor.

From FIGS. 14C and 15C, it was shown that in the group in which the composite value of IL-1β and IL-1 RAP and the composite value of IL-1β and IL-1 R2 before starting treatment were higher than the cutoff value, the progression-free survival rate for all cases was significantly higher as compared with the group in which the composite values before starting treatment were lower than the cutoff value. From these results, it was shown that the prognosis of cancer can be predicted using the composite value of IL-1β and IL-1RAP and the composite value of IL-1β and IL-1R2 in blood (serum) before starting treatment as an indicator.

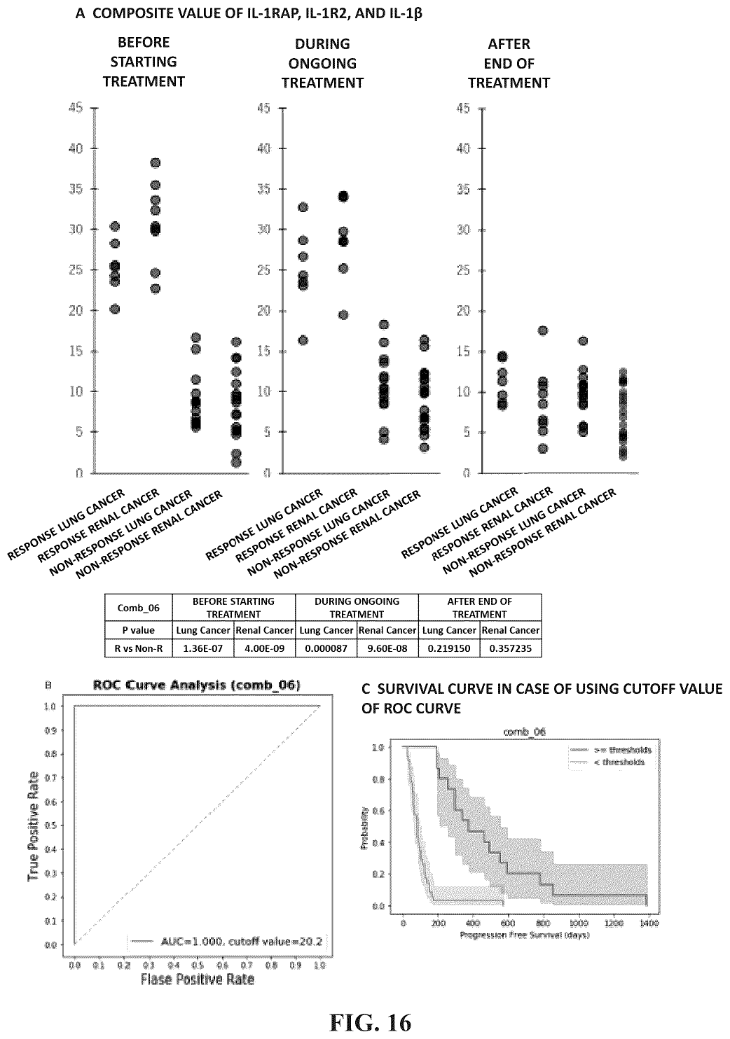

From FIG. 16A, it was shown that the indicator obtained by linearly combining the concentrations of IL-1 RAP, IL-1 R2, and IL-1β in the serum (0.0824×IL1RAP+1.2269×IL1-R2−2.7216×IL-1β) was significantly higher in the response group than in the non-response group from before starting treatment. From FIG. 16B, as a result of ROC analysis using the composite value of IL-1RAP, IL-1R2, and IL-1β, it was shown that the composite value of IL-TRAP, IL-1R2, and IL-1β before starting treatment can completely separate the response group and the non-response group (Table 8). From these results, it was shown that the composite value of IL-TRAP, IL-1R2, and IL-1β in blood (serum) before starting treatment can predict responsiveness to an immune checkpoint inhibitor.

From FIG. 16A, it was also shown that the composite value of IL-TRAP, IL-1R2, and IL-1β was significantly higher in the response group than in the non-response group even after starting treatment (one point during ongoing treatment). From this result, it was shown that the composite value of IL-1RAP, IL-1R2, and IL-1β in blood (serum) after starting treatment can predict responsiveness to an immune checkpoint inhibitor.

From FIG. 16A, it also became clear that the composite value of IL-1RAP, IL-1R2, and IL-1β decreased to the same level as that in the non-response group at a stage where exacerbation of tumor was observed and therapeutic resistance to an immune checkpoint inhibitor was shown (timing of invalidation) in the response group. From this result, it was shown that the composite value of IL-1RAP, IL-1R2, and IL-1β in blood (serum) after starting treatment can predict responsiveness (including non-responsiveness, that is, therapeutic resistance and invalidation of an immune checkpoint inhibitor) to an immune checkpoint inhibitor.

From FIG. 16C, it was shown that in the group in which the composite value of IL-1RAP, IL-1R2, and IL-1β before starting treatment was higher than the cutoff value, the progression-free survival rate for all cases was significantly higher as compared with the group in which the composite value before starting treatment was lower than the cutoff value. From this result, it was shown that the composite value of IL-1RAP, IL-1R2, and IL-1β in blood (serum) before starting treatment can predict the prognosis of cancer.

### Example 7: IL-1R1 Correlates with Responsiveness to Immune Checkpoint Inhibitor

A clinical study was conducted to determine the correlation of IL-1β as the IL-1 signaling pathway molecule with responsiveness to an immune checkpoint inhibitor.

### (1) Clinical Observation Test

The clinical observation test was performed in the same manner as in (1) of Example 3.

### (2) Measurement of Serum Protein Concentration

The human IL-1R1 concentration was measured using Human IL-1R1 DuoSet ELISA (Catalog No. DY269, R&D Systems) according to the protocol.

### (3) ROC Analysis

ROC analysis was performed in the same manner as in (3) of Example 4.

### (4) Analysis of Progression-Free Survival Rate

The progression-free survival rate was performed in the same manner as in (4) of Example 4.

TABLE 9
Therapeutic responsiveness prediction accuracy for immune checkpoint
inhibitor-administered patients in case of using cutoff value
of ROC curve of IL-1R1 concentration before starting treatment

| | All cases | Lung cancer | Renal cancer |
|---|---|---|---|
| Sensitivity | 0.812500 | 1.000000 | 0.666667 |
| Specificity | 1.000000 | 1.000000 | 1.000000 |
| Positive predictive value | 1.000000 | 1.000000 | 1.000000 |
| Negative predictive value | 0.918919 | 1.000000 | 0.869565 |

TABLE 10
Therapeutic responsiveness prediction accuracy for immune checkpoint
inhibitor-administered patients in case of using cutoff value
of ROC curve of composite value of IL-1R1 concentration and
IL-1RAP concentration before starting treatment

| | All cases | Lung cancer | Renal cancer |
|---|---|---|---|
| Sensitivity | 0.937500 | 1.000000 | 0.888889 |
| Specificity | 1.000000 | 1.000000 | 1.000000 |
| Positive predictive value | 1.000000 | 1.000000 | 1.000000 |
| Negative predictive value | 0.971429 | 1.000000 | 0.952381 |

TABLE 11
Therapeutic responsiveness prediction accuracy for immune
checkpoint inhibitor-administered patients in case
of using cutoff value of ROC curve of composite value
of IL-1R1 and IL-1R2 before starting treatment

| | All cases | Lung cancer | Renal cancer |
|---|---|---|---|
| Sensitivity | 0.937500 | 1.000000 | 0.888889 |
| Specificity | 1.000000 | 1.000000 | 1.000000 |
| Positive predictive value | 1.000000 | 1.000000 | 1.000000 |
| Negative predictive value | 0.971429 | 1.000000 | 0.952381 |

TABLE 12
Therapeutic responsiveness prediction accuracy for immune
checkpoint inhibitor-administered patients in case
of using cutoff value of ROC curve of composite value
of IL-1R1 and IL-1β before starting treatment

| | All cases | Lung cancer | Renal cancer |
|---|---|---|---|
| Sensitivity | 0.937500 | 1.000000 | 0.888889 |
| Specificity | 1.000000 | 1.000000 | 1.000000 |
| Positive predictive value | 1.000000 | 1.000000 | 1.000000 |
| Negative predictive value | 0.971429 | 1.000000 | 0.952381 |

TABLE 13
Therapeutic responsiveness prediction accuracy for immune
checkpoint inhibitor-administered patients in case of
using cutoff value of ROC curve of composite value of
IL-1R1, IL-1RAP, and IL-1β before starting treatment

| | All cases | Lung cancer | Renal cancer |
|---|---|---|---|
| Sensitivity | 0.937500 | 1.000000 | 0.888889 |
| Specificity | 1.000000 | 1.000000 | 1.000000 |
| Positive predictive value | 1.000000 | 1.000000 | 1.000000 |
| Negative predictive value | 0.971429 | 1.000000 | 0.952381 |

TABLE 14
Therapeutic responsiveness prediction accuracy for immune
checkpoint inhibitor-administered patients in case of
using cutoff value of ROC curve of composite value of
IL-1R1, IL-1R2, and IL-1β before starting treatment

| | All cases | Lung cancer | Renal cancer |
|---|---|---|---|
| Sensitivity | 1.000000 | 1.000000 | 1.000000 |
| Specificity | 0.970588 | 0.928571 | 1.000000 |
| Positive predictive value | 0.941177 | 0.875000 | 1.000000 |
| Negative predictive value | 1.000000 | 1.000000 | 1.000000 |

TABLE 15
Therapeutic responsiveness prediction accuracy for immune
checkpoint inhibitor-administered patients in case of
using cutoff value of ROC curve of composite value of
IL-1R1, IL-1R2, and IL-1RAP before starting treatment

| | All cases | Lung cancer | Renal cancer |
|---|---|---|---|
| Sensitivity | 1.000000 | 1.000000 | 1.000000 |
| Specificity | 1.000000 | 1.000000 | 1.000000 |
| Positive predictive value | 1.000000 | 1.000000 | 1.000000 |
| Negative predictive value | 1.000000 | 1.000000 | 1.000000 |

TABLE 16
Therapeutic responsiveness prediction accuracy for immune
checkpoint inhibitor-administered patients in case of using
cutoff value of ROC curve of composite value of IL-1R1,
IL-1R2, IL-1RAP, and IL-1β before starting treatment

| | All cases | Lung cancer | Renal cancer |
|---|---|---|---|
| Sensitivity | 1.000000 | 1.000000 | 1.000000 |
| Specificity | 1.000000 | 1.000000 | 1.000000 |
| Positive predictive value | 1.000000 | 1.000000 | 1.000000 |
| Negative predictive value | 1.000000 | 1.000000 | 1.000000 |

### (5) Results

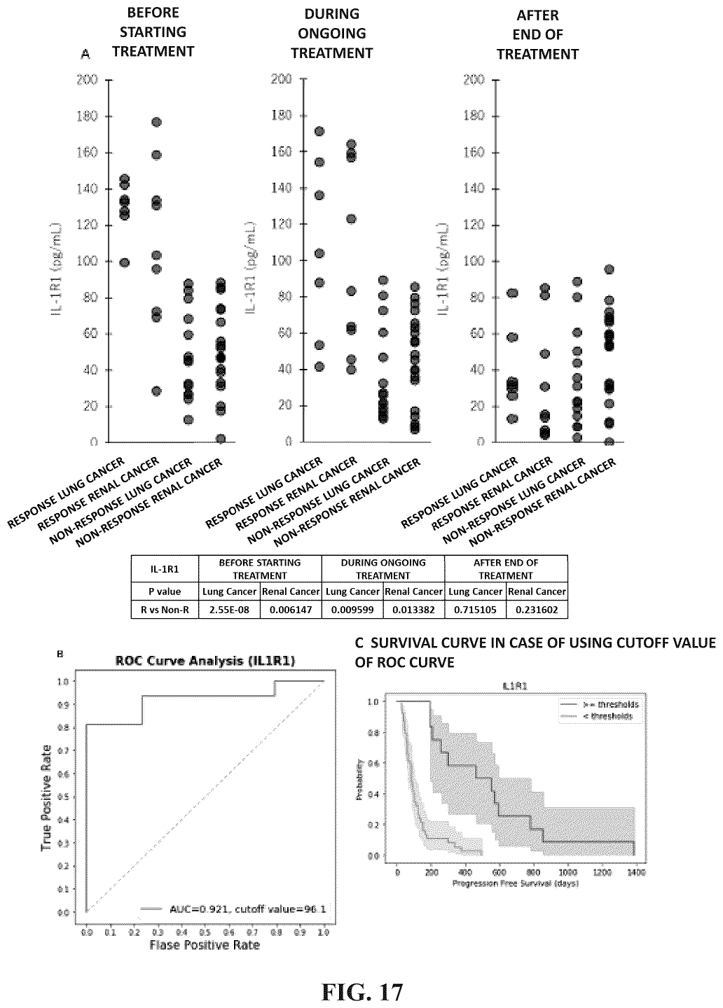

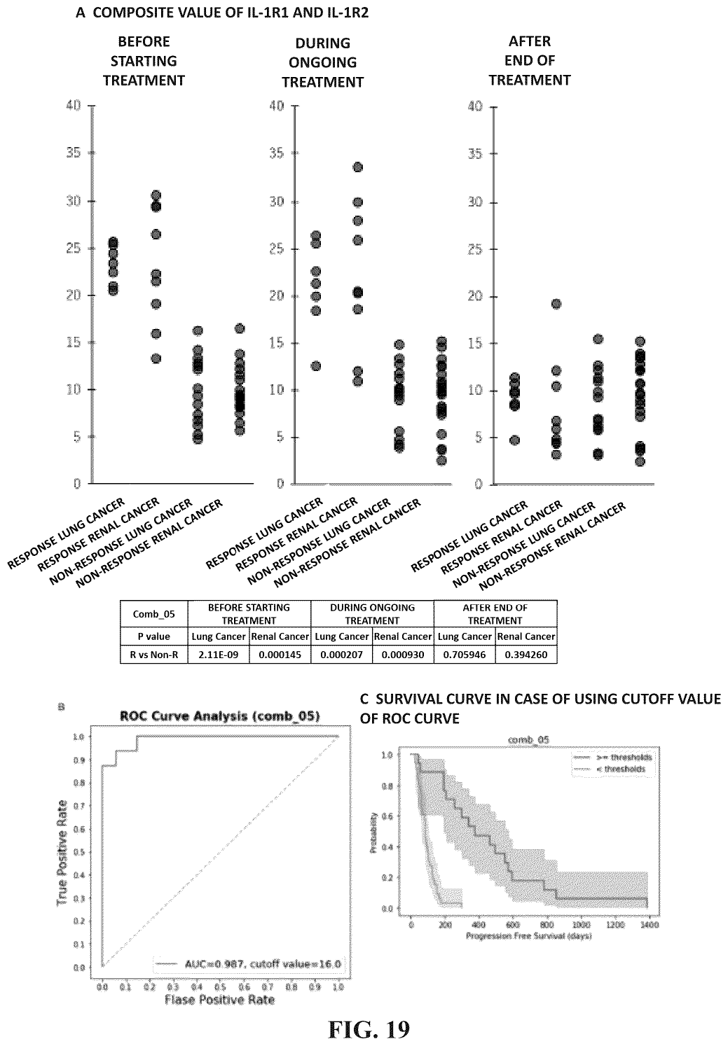

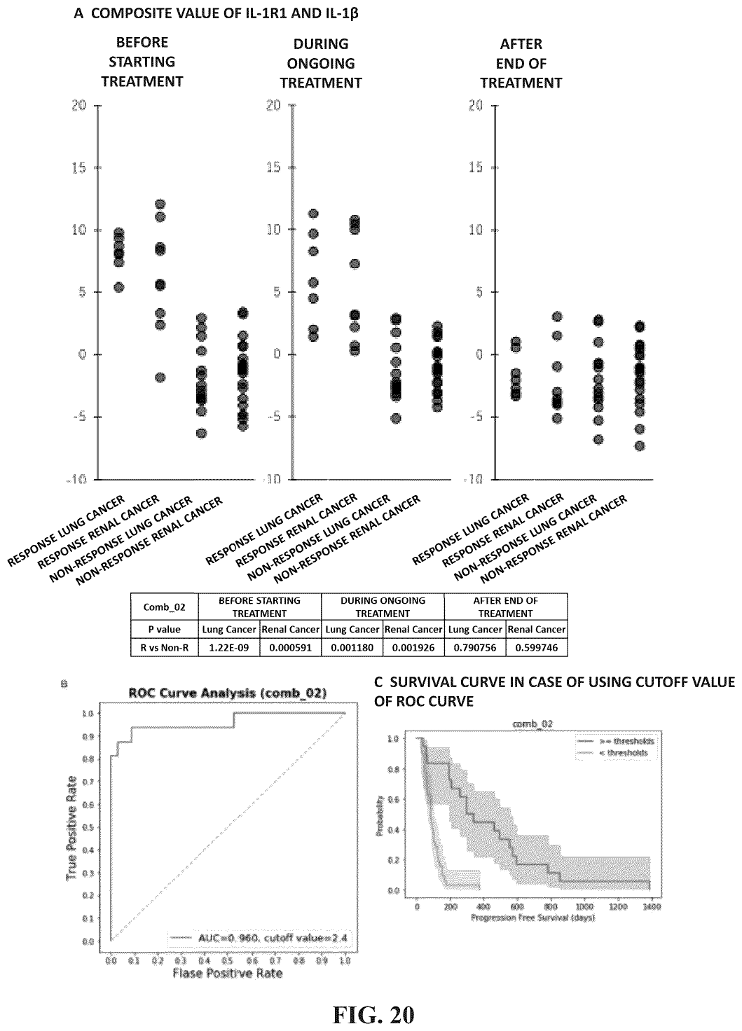

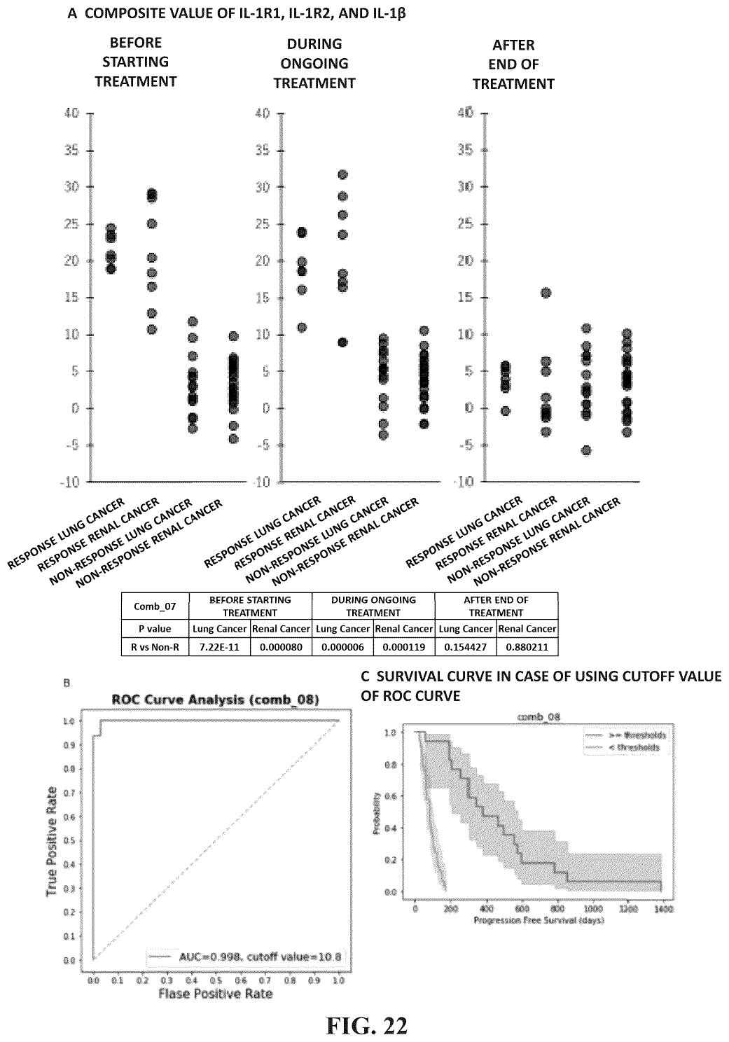

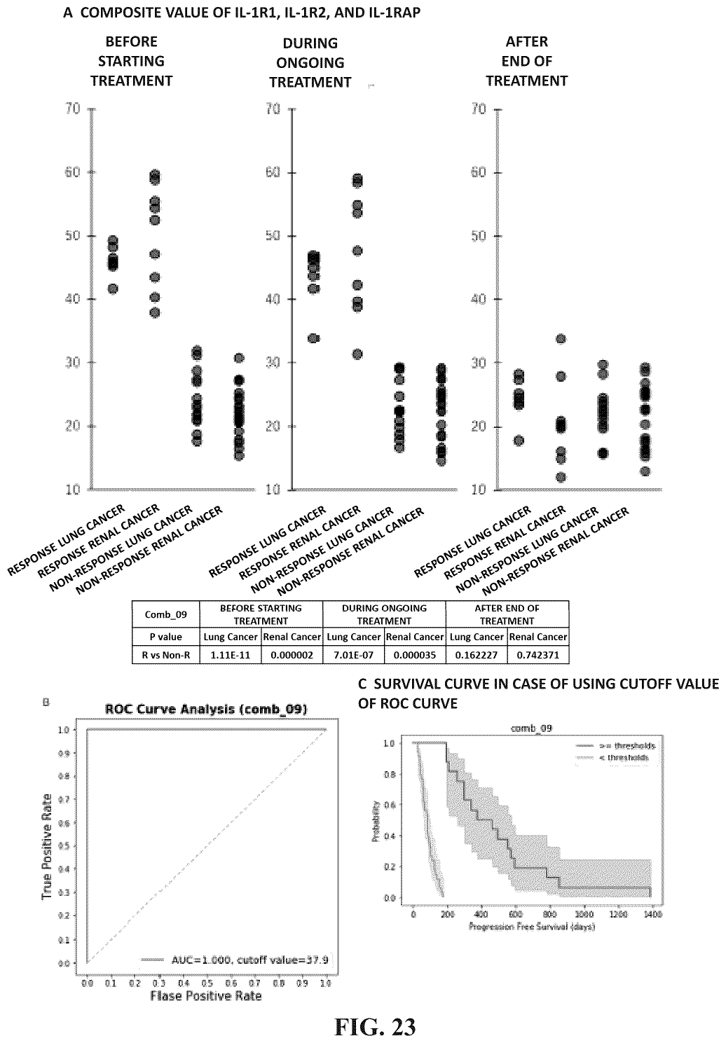

The results were as shown in Tables 9 to 16 and FIGS. 17 to 24.

From FIG. 17A, it was shown that the IL-1R1 concentration was significantly higher in the response group than in the non-response group from before starting treatment. From FIG. 17B, as a result of ROC analysis using the IL-1 R1 concentration before starting treatment, it was shown that the IL-1R1 concentration before starting treatment can accurately separate the response group and the non-response group (Table 9). From these results, it was shown that responsiveness to an immune checkpoint inhibitor can be predicted using the IL-1R1 concentration in blood (serum) before starting treatment as an indicator.

From FIG. 17A, it was also shown that the IL-1R1 concentration was significantly higher in the response group than in the non-response group even after starting treatment (one point during ongoing treatment). From this result, it was shown that responsiveness to an immune checkpoint inhibitor can be predicted using the IL-1β concentration in blood (serum) after starting treatment as an indicator.

From FIG. 17A, it also became clear that the IL-1R1 concentration decreased to the same level as that in the non-response group at a stage where exacerbation of tumor was observed and therapeutic resistance to an immune checkpoint inhibitor was shown (timing of invalidation) in the response group. From this result, it was shown that responsiveness (including non-responsiveness, that is, therapeutic resistance and invalidation of an immune checkpoint inhibitor) to an immune checkpoint inhibitor can be predicted using the IL-1R1 concentration in blood (serum) after starting treatment as an indicator.

From FIG. 17C, it was shown that in the group in which the IL-1R1 concentration before starting treatment was higher than the cutoff value, the progression-free survival rate for all cases was significantly higher as compared with the group in which the IL-1R1 concentration before starting treatment was lower than the cutoff value. From these results, it was shown that the prognosis of cancer can be predicted using the IL-1R1 concentration in blood (serum) before starting treatment as an indicator.

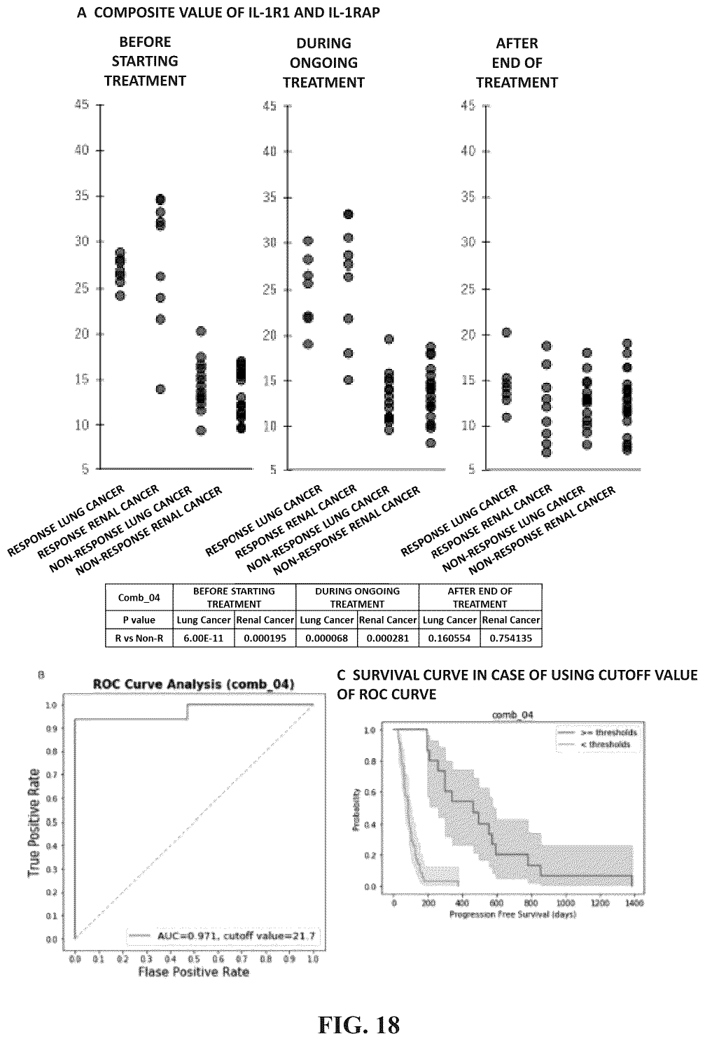

From FIGS. 18A, 19A, and 20A, it was shown that the indicator obtained by linearly combining the concentrations of IL-1β and IL-TRAP in the serum (0.1062×IL-1R1+0.082×IL-1RAP), the indicator obtained by linearly combining the concentrations of IL-1R1 and IL-1R2 in the serum (0.0856×IL-1R1+0.9566×IL-1R2), and the indicator obtained by linearly combining the concentrations of IL-1R1 and IL-1β in the serum (0.0826×IL-1β−2.019 k IL-1β) were significantly higher in the response group than in the non-response group from before starting treatment. From FIGS. 18B, 19B, and 20B, as a result of ROC analysis using the composite value of IL-1R1 and IL-1RAP, the composite value of IL-1R1 and IL-1R2, and the composite value of IL-1R1 and IL-1β before starting treatment, the composite value of IL-1R1 and IL-1 RAP, the composite value of IL-1β and IL-1R2, and the composite value of IL-1R1 and IL-1β before starting treatment can accurately separate the response group and the non-response group (Tables 10 to 12). From these results, it was shown that the composite value of IL-1R1 and IL-1RAP, the composite value of IL-1R1 and IL-1R2, and the composite value of IL-1R1 and IL-1β in blood (serum) before starting treatment can predict responsiveness to an immune checkpoint inhibitor.

From FIGS. 18A, 19A, and 20A, it was also shown that the composite value of IL-1β and IL-1RAP, the composite value of IL-1R1 and IL-1R2, and the composite value of IL-1R1 and IL-1β were significantly higher in the response group than in the non-response group even after starting treatment (one point during ongoing treatment). From this result, it was shown that the composite value of IL-1R1 and IL-1RAP, the composite value of IL-1R1 and IL-1R2, and the composite value of IL-1R1 and IL-1β in blood (serum) after starting treatment can predict responsiveness to an immune checkpoint inhibitor.

From FIGS. 18A, 19A, and 20A, it also became clear that the composite value of IL-1R1 and IL-1RAP, the composite value of IL-1R1 and IL-1R2, and the composite value of IL-1R1 and IL-1β decreased to the same level as that in the non-response group at a stage where exacerbation of tumor was observed and therapeutic resistance to an immune checkpoint inhibitor was shown (timing of invalidation) in the response group. From this result, it was shown that the composite value of IL-1R1 and IL-TRAP, the composite value of IL-1R1 and IL-1R2, and the composite value of IL-1R1 and IL-1β in blood (serum) after starting treatment can predict responsiveness (including non-responsiveness, that is, therapeutic resistance and invalidation of an immune checkpoint inhibitor) to an immune checkpoint inhibitor.

From FIGS. 18C, 19C, and 20C, it was shown that in the group in which the composite value of IL-1R1 and IL-1 RAP, the composite value of IL-1R1 and IL-1R2, and the composite value of IL-1R1 and IL-1β before starting treatment were higher than the cutoff value, the progression-free survival rate for all cases was significantly higher as compared with the group in which the composite values before starting treatment were lower than the cutoff value. From this result, it was shown that the composite value of IL-1R1 and IL-1RAP, the composite value of IL-1β and IL-1R2, and the composite value of IL-1R1 and IL-1β in blood (serum) before starting treatment can predict the prognosis of cancer.

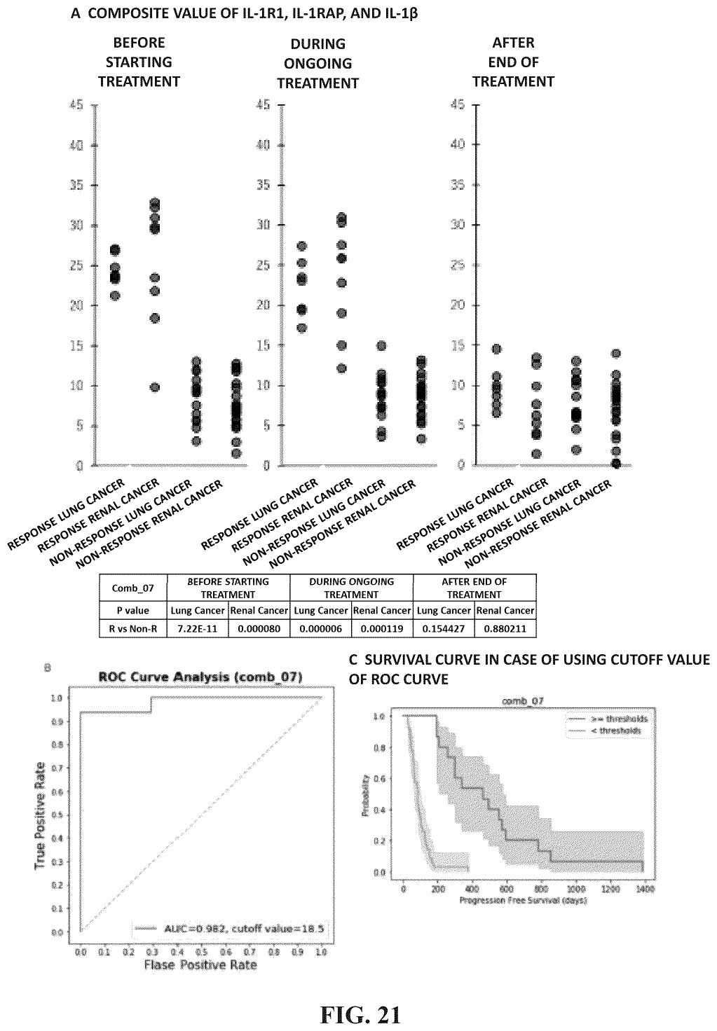

From FIGS. 21A, 22A, and 23A, it was shown that the indicator obtained by linearly combining the concentrations of IL-1R1, IL-1 RAP, and IL-1β in the serum (0.1061×IL-1R1+0.0835 k IL-1RAP−2.1135×IL-1β), the indicator obtained by linearly combining the concentrations of IL-1R1, IL-1R2, and IL-1β in the serum (0.0853×IL-1R1+1.0615×IL-1R2−2.5217×IL-1β), and the indicator obtained by linearly combining the concentrations of IL-1R1, IL-1R2, and IL-1 RAP in the serum (0.1146×IL-1R1+1.1828×IL-1R2+0.1007×IL-1 RAP) were significantly higher in the response group than in the non-response group from before starting treatment. From FIGS. 21B, 22B, and 23B, as a result of ROC analysis using the composite value of IL-1R1, IL-1RAP, and IL-1β, the composite value of IL-1R1, IL-1R2, and IL-1β, and the composite value of IL-1R1, IL-1R2, and IL-1 RAP before starting treatment, the composite value of IL-1R1, IL-1RAP, and IL-1β, the composite value of IL-1R1, IL-1R2, and IL-1β, and the composite value of IL-1 R1, IL-1R2, and IL-1RAP before starting treatment can accurately separate the response group and the non-response group (Tables 13 to 15). From these results, it was shown that the composite value of IL-1 R1, IL-TRAP, and IL-1β, the composite value of IL-1R1, IL-1R2, and IL-1β, and the composite value of IL-1R1, IL-1R2, and IL-TRAP in blood (serum) before starting treatment can predict responsiveness to an immune checkpoint inhibitor.

From FIGS. 21A, 22A, and 23A, it was also shown that the composite value of IL-1R1, IL-1RAP, and IL-1β, the composite value of IL-1R1, IL-1R2, and IL-1β, and the composite value of IL-1R1, IL-1R2, and IL-TRAP were significantly higher in the response group than in the non-response group even after starting treatment (one point during ongoing treatment). From this result, it was shown that the composite value of IL-1R1, IL-1RAP, and IL-1β, the composite value of IL-1R1, IL-1R2, and IL-1β, and the composite value of IL-1R1, IL-1R2, and IL-1RAP in blood (serum) after starting treatment can predict responsiveness to an immune checkpoint inhibitor.

From FIGS. 21A, 22A, and 23A, it also became clear that the composite value of IL-1R1, IL-1RAP, and IL-1β, the composite value of IL-1R1, IL-1R2, and IL-1β, and the composite value of IL-1R1, IL-1R2, and IL-1 RAP decreased to the same level as that in the non-response group at a stage where exacerbation of tumor was observed and therapeutic resistance to an immune checkpoint inhibitor was shown (timing of invalidation) in the response group. From this result, it was shown that the composite value of IL-1R1, IL-1RAP, and IL-1β, the composite value of IL-1R1, IL-1R2, and IL-1β, and the composite value of IL-1R1, IL-1R2, and IL-1RAP in blood (serum) after starting treatment can predict responsiveness (including non-responsiveness, that is, therapeutic resistance and invalidation of an immune checkpoint inhibitor) to an immune checkpoint inhibitor.

From FIGS. 21C, 22C, and 23C, it was shown that in the group in which the composite value of IL-1R1, IL-1RAP, and IL-1β, the composite value of IL-1R1, IL-1R2, and IL-1β, and the composite value of IL-1R1, IL-1R2, and IL-1RAP before starting treatment were higher than the cutoff value, the progression-free survival rate for all cases was significantly higher as compared with the group in which the composite values before starting treatment were lower than the cutoff value. From this result, it was shown that the composite value of IL-1R1, IL-1RAP, and IL-1β, the composite value of IL-1R1, IL-1R2, and IL-1β, and the composite value of IL-1R1, IL-1R2, and IL-1RAP in blood (serum) before starting treatment can predict the prognosis of cancer.

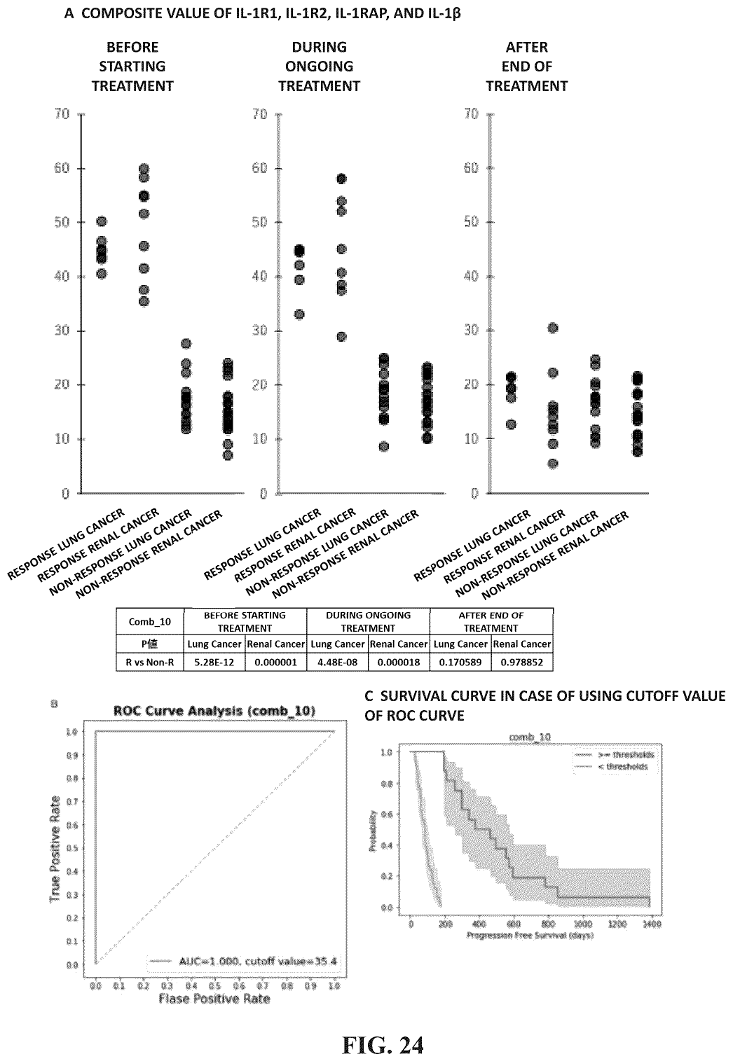

From FIG. 24A, it was shown that the indicator obtained by linearly combining the concentrations of IL-1R1, IL-1R2, IL-1RAP, and IL-1β in the serum (0.11154×IL-1R1+1.30615×IL-1R2+0.1045×IL-1RAP−2.756×IL-1β) was significantly higher in the response group than in the non-response group from before starting treatment. From FIG. 24B, as a result of ROC analysis using the composite value of IL-1R1, IL-1R2, IL-1RAP, and IL-1β before starting treatment, it was shown that the composite value of IL-1R1, IL-1R2, IL-1RAP, and IL-1β before starting treatment can completely separate the response group and the non-response group (Table 16). From these results, it was shown that the composite value of IL-1R1, IL-1R2, IL-1RAP, and IL-1β in blood (serum) before starting treatment can predict responsiveness to an immune checkpoint inhibitor.

From FIG. 24A, it was also shown that the composite value of IL-1R1, IL-1R2, IL-1RAP, and IL-1β was significantly higher in the response group than in the non-response group even after starting treatment (one point during ongoing treatment). From this result, it was shown that the composite value of IL-1R1, IL-1R2, IL-TRAP, and IL-1β in blood (serum) after starting treatment can predict responsiveness to an immune checkpoint inhibitor.

From FIG. 24A, it also became clear that the composite value of IL-1R1, IL-1R2, IL-TRAP, and IL-1β decreased to the same level as that in the non-response group at a stage where exacerbation of tumor was observed and therapeutic resistance to an immune checkpoint inhibitor was shown (timing of invalidation) in the response group. From this result, it was shown that the composite value of IL-1R1, IL-1R2, IL-1RAP, and IL-1β in blood (serum) after starting treatment can predict responsiveness (including non-responsiveness, that is, therapeutic resistance and invalidation of an immune checkpoint inhibitor) to an immune checkpoint inhibitor.

From FIG. 24C, it was shown that in the group in which the composite value of IL-1R1, IL-1R2, IL-1RAP, and IL-1β before starting treatment was higher than the cutoff value, the progression-free survival rate for all cases was significantly higher as compared with the group in which the composite value before starting treatment was lower than the cutoff value. From this result, it was shown that the composite value of IL-1R1, IL-1R2, IL-1RAP, and IL-1β in blood (serum) before starting treatment can predict the prognosis of cancer.

1. A method for predicting responsiveness to an immune checkpoint inhibitor, the method comprising predicting therapeutic responsiveness of a subject in need of treatment of cancer to an immune checkpoint inhibitor by using an amount or concentration of an IL-1 signaling pathway molecule in a biological sample of the subject as an indicator.

2. The method according to claim 1, comprising a step of measuring the amount or concentration of the IL-1 signaling pathway molecule in the biological sample of the subject.

3. The method according to claim 1, comprising a step of comparing the amount or concentration of the IL-1 signaling pathway molecule in the biological sample of the subject with a cutoff value.

4. The method according to claim 1, wherein the IL-1 signaling pathway molecule is one or two or more substances (IL-1 signaling pathway molecule (a)) selected from the group consisting of (1) IL-1 RAP, (2) IL-1R2, (3) IL-1R1, (4) ST2 (IL-1RL1), and (5) IL-1Rrp2.

5. The method according to claim 4, wherein the amount or concentration of the IL-1 signaling pathway molecule (a) in the biological sample of the subject before or after starting treatment with an immune checkpoint inhibitor being higher than a cutoff value indicates that the subject is responsive to the immune checkpoint inhibitor.

6. The method according to claim 1, wherein the IL-1 signaling pathway molecule is one or two or more substances (IL-1 signaling pathway molecule (b)) selected from the group consisting of (11) IL-1β, (12) IL-1α, (13) IL-1Ra, (14) IL-33, (15) IL-38, (16) IL-36α, (17) IL-36β, (18) IL-36γ, and (19) IL-36Ra.

7. The method according to claim 6, wherein the amount or concentration of the IL-1 signaling pathway molecule (b) in the biological sample of the subject before or after starting treatment with an immune checkpoint inhibitor being lower than a cutoff value indicates that the subject is responsive to the immune checkpoint inhibitor.

8. The method according to claim 1, wherein the IL-1 signaling pathway molecule is two or more substances selected from the group consisting of (1) IL-1RAP, (2) IL-1R2, (3) IL-1R1, (4) ST2 (IL-1RL1), (5) IL-1Rrp2, (11) IL-1β, (12) IL-1α, (13) IL-1Ra, (14) IL-33, (15) IL-38, (16) IL-36α, (17) IL-36β, (18) IL-36γ, and (19) IL-36Ra.

9. The method according to claim 8, wherein one composite value calculated from a measured value of the amount or concentration of two or more IL-1 signaling pathway molecules in the biological sample of the subject before or after starting treatment with an immune checkpoint inhibitor being higher or lower than a cutoff value indicates that the subject is responsive to the immune checkpoint inhibitor.

10. The method according to claim 1, wherein the biological sample is a blood sample.

11. The method according to claim 1, which is a biological sample analysis method for predicting responsiveness to an immune checkpoint inhibitor.

12. A method for predicting prognosis of a subject suffering from cancer who has received treatment with an immune checkpoint inhibitor, the method comprising predicting the prognosis by using an amount or concentration of an IL-1 signaling pathway molecule in a biological sample of the subject as an indicator.

13. The method according to claim 12, comprising a step of measuring the amount or concentration of the IL-1 signaling pathway molecule in the biological sample of the subject.

14. The method according to claim 12 or 13, comprising a step of comparing the amount or concentration of the IL-1 signaling pathway molecule in the biological sample of the subject with a cutoff value.

15. (canceled)

16. A prediction kit of responsiveness to an immune checkpoint inhibitor or a prediction kit of prognosis of a subject suffering from cancer who has received treatment with an immune checkpoint inhibitor, comprising a means for quantifying an amount or concentration of an IL-1 signaling pathway molecule in a biological sample.

17. A method for treating cancer in a subject predicted to be responsive to treatment with an immune checkpoint inhibitor, the method comprising selecting the subject by the method according to claim 1, and subjecting the selected subject to treatment with the immune checkpoint inhibitor.

18. A method for treating cancer in a subject undergoing treatment with an immune checkpoint inhibitor, the method comprising selecting a subject predicted to be non responsive to treatment with the immune checkpoint inhibitor by the method according to claim 1 and subjecting the selected subject to treatment other than the treatment with the immune checkpoint inhibitor.
